(19) United States
(12) Patent Application Publication
(10) Pub. No.: US 2007/0028316 A1
(43) Pub. Date: Feb. 1, 2007

(54) Transgenic non-human Act1-deficient mammals and uses thereof

(76) Inventors: Xiaoxia Li, Solon, OH (US); Youcun Qian, Cleveland, OH (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/446,450

(22) Filed: Jun. 2, 2006

Related U.S. Application Data
(60) Provisional application No. 60/686,767, filed on Jun. 2, 2005.

(51) Int. Cl. A01K 67/027

(52) U.S. Cl. 800/18

(57) ABSTRACT

The present invention provides a transgenic non-human mammal which lacks a functional Act1 gene referred to herein as a “transgenic non-human Act1 knockout mammal” or a “Act1 knockout mammal”. In a particular embodiment, the genome of the Act1 knockout mammal comprises at least one non-functional allele for the endogenous Act1 gene. Thus, the invention provides a source of cells (for example, tissue, cells, cellular extracts, organelles) and transgenic non-human mammals useful for studying Act1. Further aspects of the invention provide methods for producing transgenic non-human Act1 knockout mammals and transgenic non-human Act1 deficient mammals produced by the methods; targeting vectors for use in producing Act1 deficient mammals; methods for the identification of agents (for example, diagnostic or therapeutic agents) which inhibit or mimic Act1 activity; and methods of identifying agents that can be used to treat and/or prevent a disease or condition associated with a defect in Act1 (e.g., systemic lupus erythematosus (SLE); Sjögren's syndrome, cancer).

### RELATED APPLICATION(S)

This application claims the benefit of U.S. Provisional Application No. 60/686,767 filed Jun. 2, 2005.

The entire teachings of the above application(s) are incorporated herein by reference.

### GOVERNMENT SUPPORT

The invention was supported, in whole or in part, by a grant GM 600020 from the National Institutes of Health. The Government has certain rights in the invention.

### BACKGROUND OF THE INVENTION

TNF receptor (TNFR) superfamily members, CD40 and BAFFR play critical roles in B cell survival and differentiation. A greater understanding of the signaling pathways mediated by CD40 and BAFF is needed.

### SUMMARY OF THE INVENTION

The present invention is directed to a transgenic non-human mammal (e.g., a mouse) whose genome comprises a disruption of an Act1 gene. In one embodiment, the transgenic non-human mammal has lymphoid system defects comprising lymphadenopathy, hypergammaglobulinemia and production of autoantibodies. In another embodiment, the disruption of the Act1 gene is in exon 2 of the Act 1 gene (e.g., a deletion of exon 2) of the transgenic non-human mammal. In a particular embodiment, an Act1 gene targeting vector is used to delete exon 2 of the Act1 gene. A particular example of an Act1 gene targeting vector comprises in a 5′ to 3′ order: a first intron of the Act1 gene—a recombination site—a marker gene—exon 2 of the Act1 gene—a recombination site—a second intron of the Act1 gene. The recombination site can be, for example, a loxP site and the marker gene can be, for example, a Neo gene.

The present invention is also directed to a method of producing a transgenic non-human mammal whose genome comprises a homozygous disruption of an Act1 gene comprising introducing a targeting vector which disrupts the Act1 gene into an embryonic stem cell, thereby producing a transgenic embryonic stem cell with a disrupted Act1 gene. The transgenic embryonic stem cell is then introduced into a blastocyte, thereby forming a chimeric blastocyte, and the chimeric blastocyte is introduced into the uterus of a pseudo-pregnant non-human mammal under conditions in which the pseudo-pregnant non-human mammal gives birth to transgenic non-human mammals whose genome comprises a heterozygous disruption of the Act1 gene. The transgenic non-human mammals are bred under conditions in which a transgenic non-human mammal whose genome comprises a homozygous disruption the Act1 gene is produced. The invention also encompasses transgenic non-human mammals produced by the methods described herein.

Isolated cells having a genome comprising a disruption of an Act1 gene, wherein the cell is isolated from a transgenic non-human mammal comprising a disruption of the Act1 gene are also encompassed by the present invention.

In particular embodiments the present invention is directed to a transgenic non-human mammal whose genome comprises a disruption of an Act1 gene wherein the disruption of the Act1 gene is specific to B cells of the transgenic non-human mammal. In another embodiment, the present invention is directed to a transgenic non-human mammal whose genome comprises a disruption of an Act1 gene wherein the disruption of the Act1 gene is specific to epithelial cells of the transgenic non-human mammal.

In another embodiment, the invention pertains to a transgenic non-human mammal whose genome comprises a disruption of a CD40 gene and an Act1 gene. Such transgenic non-human mammals have enlarged lymph nodes and an enlarged spleen.

A transgenic non-human mammal whose genome comprises a disruption of a BAFF gene and an Act1 gene is also encompassed by the present invention. Such transgenic non-human mammals have enlarged lymph nodes and an enlarged spleen.

The present invention is also directed to a transgenic non-human mammal whose genome comprises a heterozygous disruption of an Act1 gene wherein disruption of the Act1 gene in a homozygous states results in a transgenic mouse having lymphoid system defects comprising lymphadenopathy, hypergammaglobulinemia and production of auto antibodies.

Methods of producing antibodies (e.g., polyclonal, monoclonal) which specifically bind to an antigen are also provided. In one embodiment, the method comprises introducing the antigen into a transgenic non-human mammal whose genome comprises a disruption of an Act1 gene under conditions in which antibodies which specifically bind to the antigen are produced in the mammal. The method can further comprise isolating the antibodies which specifically bind to the antigen from the transgenic non-human mammal. The antigen for use in the methods can be, for example, a T cell independent antigen and/or a T cell dependent antigen. In particular embodiments, the antigen does not initiate a strong antibody response in a wild type mammal. In yet another embodiment, the antibodies produced by the method have a higher affinity for the antigen when compared to antibodies made in a wild type non-human mammal. The invention is also directed to antibodies produced by the methods described herein.

The present invention is also directed to methods of producing a hybridoma which expresses a monoclonal antibody that is specifically directed to an antigen. In one embodiment, the method comprises introducing the antigen into a transgenic non-human mammal whose genome comprises a disruption of an Act1 gene, under conditions in which antibodies which specifically bind to the antigen are produced in the mammal. B cells from the mammal are isolated and a B cell (one or more) which expresses an antibody that specifically recognizes the antigen is selected. The selected B cell is fused with an immortal cell, thereby producing a hybridoma which expresses a monoclonal antibody that is specifically directed to the antigen. The method can further comprise isolating the hybridoma (one or more). The invention is also directed to hybridomas produced by the methods described herein.

Methods of producing a monoclonal antibody that is specifically directed to an antigen are also encompassed by the present invention. The method comprises introducing the antigen into a transgenic non-human mammal whose genome comprises a disruption of an Act1 gene, under conditions in which antibodies which specifically bind to the antigen are produced in the mammal. B cells from the mammal are isolated and a B cell (one or more) which expresses an antibody that specifically recognizes the antigen is selected. The selected B cell is fused with an immortal cell, thereby producing a hybridoma (one or more) which expresses a monoclonal antibody that is specifically directed to the antigen. The hybridoma is maintained under conditions in which the monoclonal antibody is expressed, thereby producing a monoclonal antibody that is specifically directed to an antigen. The method can further comprise isolating the monoclonal antibody (one or more). The invention is also directed to monoclonal antibodies produced by the methods described herein.

The present invention is also directed to methods of identifying an agent to treat or prevent systemic lupus erythematosus (SLE). In one embodiment, the method comprises administering to a transgenic non-human mammal whose genome comprises a disruption of an Act1 gene an agent to be assessed. The ability of the agent to treat or prevent SLE in the transgenic non-human mammal is assessed, wherein if the agent treats or prevents SLE in the transgenic non-human mammal, then the agent is an agent which can be used to treat or prevent SLE.

Also encompassed by the present invention are methods of identifying an agent to treat or prevent Sjögren's syndrome. In one embodiment, the method comprises administering to a transgenic non-human mammal whose genome comprises a disruption of an Act1 gene an agent to be assessed. The ability of the agent to treat or prevent Sjögren's syndrome in the transgenic non-human mammal is assessed, wherein if the agent treats or prevents Sjögren's syndrome in the transgenic non-human mammal, then the agent is an agent which can be used to treat or prevent Sjögren's syndrome.

The present invention is also directed to methods of identifying an agent to treat or prevent cancer (e.g., lung adenoma, skin fibroepithelioma). In one embodiment, the method comprises administering to a transgenic non-human mammal whose genome comprises a disruption of an Act1 gene an agent to be assessed. The ability of the agent to treat or prevent cancer in the transgenic non-human mammal is assessed, wherein if the agent treats or prevents cancer in the transgenic non-human mammal, then the agent is an agent which can be used to treat or prevent cancer.

The invention is also directed to a targeting vector which can be used to produce a transgenic non-human mammal which genome comprises a disruption of an Act1 gene. In one embodiment, the targeting vector comprises in a 5′ to 3′ order: an intron of the Act1 gene (e.g., the first intron of the Act1 gene)—a recombination site (e.g., loxP)—a marker gene (e.g., Neo)—an exon of the Act1 gene (e.g., exon 2)—a recombination site—a second intron of the Act1 gene (e.g., the second intron of the Act1 gene).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D illustrate generation of Act1-deficient mice.

FIG. 1A shows construction of the Act1 gene-targeting vector. See Experimental Procedures. The gene encoding Act1 (WT allele); the targeting vector (Targeting vector); loxP sequences (triangles), left arm (LA); right arm (RA), Diphtoxin A gene (DTA); the targeted allele with the targeting vector (floxed allele); Cre-mediated complete deletion of the Act1-null allele (Act1-null).

FIGS. 1B and 1C show southern blot analyses of DNA from Act1-deficient mice and ES cells. Genomic DNA was extracted from mouse tail tissue or ES cells, digested with HindIII or KpnI/SpeI and analyzed by Southern blot, using the two probes (prb1 and prb2) shown in FIG. 1A. Southern analysis with prb1 (digested with Kpn1/Spe1) generates a 9.0-kb for the wild-type allele and a 7.0-kb for the Act1-null allele. Southern blot with prb2 (digested with HindIII), generates a 7.0-kb for the wild-type allele and a 5.0-kb band presents the Act1-null allele.

FIG. 1D shows RNA blot analysis. Total RNA was extracted from wild-type (+/+) or Act1-deficient (−/−) splenic B cells, untreated (C1 and C2, incubated without ligand respectively for 24 h or 48 h); or treated with BAFF, CD40 antibody or LPS for 24 or 48 h. The probes are full-length mouse cDNAs (right margins: ACT1, Act1; GAPDH, glyceraldehydes phosphodehydrogenase).

FIG. 1E is a southern blot showing CD19cre mediated deletion of Act1 in B cells. Splenic B cells (CD54R/B220+) were isolated by positive enrichment using two rounds of B220+panning. The genomic DNA prepared from these B cells or mouse tails was cut with EcoRI and SpeI and subjected to southern blot analysis with a probe (prb3 in FIG. 1A). The 7.2 kb band stands for the Act1-flox allele while the 3.6 kb band for Act1-null allele. Quantification of CD19cre mediated deletion of Act1 was performed with Scion Image. The percentage of deletion was normalized for the purity of the enriched B cell population, which was between 80-85% by flow analysis. CD19cre+/−: one CD19 wild-type allele and one CD19cre transgenic allele; CD19cre−/−: two wild type CD19 alleles; Act1^{-/flox}: one Act1-null allele and one Act1-floxed allele.

FIGS. 2A-2F show lymphoid system abnormality in Act1-deficient mice.

FIG. 2A shows an enlarged cervical lymph node and spleen in Act1-deficient mice. Cervical lymph nodes and spleens dissected from wild-type (+/+) and Act1-deficient (−/−) mice were compared.

FIG. 2B shows the histology of lymph node. Frozen sections from wild-type (+/+) and Act 1-deficient (−/−) cervical lymph node were immunostained for T/B cell zones with anti-B220 (blue) and anti-CD5 (brown); for germinal centers with biotinylated-PNA (brown) and for plasma cells with anti-Syndecan-1 (brown).

FIG. 2C shows histology of spleen. Frozen sections from wild-type (+/+) and Act1-deficient (−/−) spleens were simultaneously immunostained for metallophilic macrophages and B cells with anti-MOMA1 (brown) and anti-B220 (blue). They were also immunostained for T/B cell zones with anti-B220 (blue) and anti-CD5 (brown).

FIG. 2D shows immuno-staining of trachea. Tracheas from wild-type or Act1-deficient mice were immuno-stained with anti-CD3 (brown). The brown-stained CD3+ T cells are clearly detected below the epithelium in the Act1-deficient trachea.

FIGS. 2E-2F show epidermal hyperplasia and T lymphocyte infiltration in skin sections of wild type and Act1-deficient mice. Skin sections from wild type mice (Act1+/+) or two Act 1-deficient mice (Act1−/−#5 or Act1−/−#11) were stained with hematoxylin and eosin (FIG. 2E) or with anti-CD3 (FIG. 2F). Skin in the upper left panel displays thickened and disorganized epidermis, a serious crust and lymphoid infiltrates in the dermis. In the upper right panel, the edge of an extensive area of ulceration is seen. Skin changes in the knockout mouse #5 were milder, consisting of erosive dermatitis. However, skin from wild-type control mouse is normal. (FIG. 2F) The anti-CD3 stained cells are brown.

FIGS. 3A-3E shows antibody production.

FIGS. 3A-3B shows serum immunoglobulin levels. FIG. 3A shows sera from 7-week-old Act 1-deficient mice (Act 1−/−) (n=5), wild-type littermates (Act1+/+)(n=4), CD40-deficient mice (CD40−/−)(n=6), double knockout mice of Act1 and CD40 (Act1−/−CD40−/−) (n=5), BAFF deficient mice (Baff−/−) (n=8) and double knockout mice of Act1 and BAFF (Act1−/−Baff−/−) (n=12) were analyzed by ELISA for different immunoglobulin levels, including IgM, IgG1, and IgG2a.

FIG. 3B shows sera from 8-week-old control mice (CD19^{cre}Act1^{+“F) (n=}4) and B cell specific Act1 knockout mice (CD19^{cre}Act1^{-/F}) (n=6) that were analyzed by ELISA for IgM and IgG2a levels.

FIG. 3C shows T-cell dependent immune responses. Four pairs of 7-week-old Act1-deficient and wild-type littermate (+/+) mice were immunized with 20 μg of NP28-CGG in PBS. Blood was taken at day 10 and day 14 after immunization. The sera were analyzed by ELISA for total NP-specific antibodies with NP30-BSA or NP-specific high affinity antibodies with NP4-BSA. Each sample was measured using multiple dilutions and the data presented are average of 50% of the maximum binding to NP4-BSA or NP30-BSA from four mice.

FIG. 3D shows T-cell independent immune responses. Four pairs of 7-week-old mice were immunized with 20 g/ml NP-Ficoll in PBS. Blood were taken at day 0, day 8 and day 15 and analyzed for TI antibody isotypes with capture antigen NP30-BSA by ELISA. Data are shown here from the blood at day 8 after immunization. Each sample was measured using multiple dilutions and the data presented are average of 50% of the maximum binding to NP30-BSA from four mice.

FIG. 3E shows autoantibodies in Act1-deficient mice. Levels of circulating anti-dsDNA, anti-ssDNA and anti-histone IgG autoantibodies in the serum of 7-month-old Act 1-deficient (−/−) and wild-type littermate controls (+/+) were assayed by ELISA. The units of autoantibodies are an arbitrary unit scale defined relative to a standard linear curve using dilutions of monoclonal antibodies to the appropriate nuclear antigen. BWD-1(1D12) was used to generate the anti-ds-DNA and anti-ssDNA standard curves, whereas BWH-1 (2B1) was used for the standard anti-histone curve (Kotzin, B. L., et al., J. Immunol. 133, 2554-2559 (1984); Lei, X. F., et al., J. Clin. Invest 101:1342-1353 (1998)).

FIGS. 3F-3G show antibody production antibodies. Sera from 7-week-old Act1-deficient mice (Act1−/−) (n=5), wild type littermates (Act1^{+/+}) (n=4), and CD40-deficient mice (CD40−/−) (n=6), double knockout mice of Act1 and CD40 (Act1−/−CD40−/−) (n=5), BAFF deficient mice (Baff−/−) (n=8) and double knockout mice of Act1 and BAFF (Act1−/−BAFF−/−) (n=12) were analyzed by ELISA for (FIG. 3F) IgG2b, IgG3 and IgA levels and (FIG. 3G) IgE levels.

FIG. 3H shows immunostaining of germinal centers. Frozen sections from wild type (+/+) and Act1-deficient (−/−) spleens from mice day 14 after immunization with NP-CGG were immunostained for germinal centers anti anti-PNA (brown).

FIG. 3I is a graph showing germinal center cells in the spleens. Splenocytes from 7-week-old Act 1-deficient (−/−) (n=3) and wild-type littermate control (+/+) (n=3) mice day 14 after immunization with NP-CGG were stained with anti-B220, anti-GL7 and anti-FAS, and then analyzed after electronic scatter gating. The data presented are the average number of B220+GL7+FAS+cells (germinal center cells) with standard deviation from three pairs of mice. While the number of germinal centers per area was not significantly altered in the Act1-deficient spleens as compared to wild-type littermate controls in response to T-cell dependent antigen (NP-CGG), the number of total germinal center cells (defined by B220+GL7+FAS+) was significantly increased in Act1-deficient spleens (FIGS. 3H and 3I).

FIGS. 4A-4C show FACS analysis. Splenocytes from 7-week-old Act1-deficient (−/−) (n=3) and wild-type littermate control (+/+) (n=3) mice were stained with the indicated antibodies, and then analyzed after electronic scatter gating.

FIG. 4A shows increased B220+ cells in Act1-deficient spleen.

FIG. 4B shows increased follicular (CD21^{int}CD23^{hi}) and marginal zone (CD21^{hi}CD23^{lo}) B cells in Act1-deficient spleen.

FIG. 4C shows increased transitional B cells in Act1-deficient spleen. The data are presented as percentages of total splenocytes from one pair of mice. Similar results were obtained from three pairs of mice. Since the Act1-deficient spleens were enlarged, the different B cells populations are presented as actual cell numbers in Table.

FIGS. 4D-4F show enhanced B cell survival in Act1-deficient mice. Purified splenic B cells from 7-week-old Act1-deficient (−/−) and littermate control (+/+) mice untreated or treated with CD40 antibody for 4 days. These cultured B cells were then analyzed by flow cytometry on FSC/SSC plots. Gates R1 (low FSC, apoptotic and dead cells) and R2 (high FSC, live cells) are indicated in each plot. Freshly prepared splenocytes displayed increased FSC levels (the R2 population, greater than 90%). After 4 days in culture, most splenocytes are dead as evidence by lower FSC(R1 population). The data presented is percentage of live cells from one of the three independent experiments with similar results (FIG. 4D). To confirm the R1 (apoptotic and dead) and R2 (live) populations, we used annexin V and propidium iodine (PI) to stain the apoptotic and dead cells respectively. FIGS. 4E and 4F are graphs of B cell proliferation (FIG. 4E) and T cell proliferation (FIG. 4F). 2×10⁵ cells/well of B220+B cells (FIG. 4E) or Thy.1+T cells (FIG. 4F) purified from spleens of Act1-deficient mice (KO, n=3) or wild-type control mice (WT, n=3) were untreated or treated with anti-CD40 or anti-CD3/anti-CD28, IL-4 for 72 hrs. Cell proliferation was assessed with thymidine incorporation (added 16 hr before harvest). CD40-mediated proliferation was increased 40% in Act1-deficient B cells as compared to that in wild-type cells, which probably contributes to the enhanced CD40-mediated B cell survival in Act1-deficient mice (FIGS. 5A-5E). As a control, it was observed that CD3/CD28-mediated T cell proliferation was the same between wildtype and Act1-deficient mice.

FIGS. 5A-5C shows B cell survival.

FIG. 5A shows enhanced B cell survival in Act1-deficient mice. Purified splenic B cells from 7-week-old Act1-deficient (−/−) and littermate control (+/+) mice untreated or treated with CD40 antibody for 4 days. These cultured B cells were then analyzed by flow cytometry on FSC/SSC plots (FIG. 4D) Annexin V staining for apoptotic cells and PI staining for dead cells were used in the FACS analysis. The data presented are the average of percentage of cell survival from three pairs of mice.

FIG. 5B shows increased percentage of live B cells from B cell specific Act1 knockout mice upon stimulation with CD40 antibody or BAFF ligand. The data are shown as the average of percentage of B cell survival from B cell specific Act1 knockout mice (CD19^{cre}Act1^{-/F}) and control mice (CD19^{cre}Act^{+/F}).

FIG. 5C shows enhanced CD40-mediated induction of Bcl-x1 in Act1-deficient splenic B cells. Splenic B cells from 7-week-old Act1-deficient (−/−) and littermate control (+/+) were untreated or treated with CD40 antibody or BAFF for 24 h or 48 h. The cell extracts were analyzed by western blot with the antibodies against Bcl-x1 and Bcl-2. α-Tubulin was used as a loading control. The Bcl-x1 levels were analyzed by Scion Image 1.62C alias and presented as relative fold of induction of the untreated samples.

FIGS. 5D-5E are gels showing gene expression of BAFF (FIG. 5D) and gene induction of CD154 (FIG. 5E). FIG. 5D shows equal amounts of total RNAs purified from spleens of Act1-deficient mice or wild-type mice that were used for quantitative RT-PCR for mBAFF or mActin (loading control). RT-PCR products were run on 1% agarose gel. FIG. 5E shows T cells purified from spleens of Act1-deficient mice or wild-type control mice that were untreated or treated with 1 μg/ml anti-CD3/CD28 for 24 hr. The cell extracts were run on 15% SDS-PAGE gel for western blot with anti-CD154 or anti-Actin (loading control).

FIGS. 6A-6E show increased CD40 and BAFF signaling in Act1-deficient splenic B cells. Splenic B cells from 7-week-old Act1-deficient (−/−) and littermate control (+/+) mice were untreated or treated with CD40 antibody, mBAFF, anti-IgM or mApril for the indicated times. The whole cell extracts were examined by western blot analysis.

FIG. 6A shows cells treated with CD40 antibody or BAFF for 3 h, 6 h or 24 h and extracts were analyzed with antibodies against P100/P52 and α-tubulin. FIG. 6B shows extracts from cells treated with CD40 antibody that were analyzed with antibodies against p-IκBα, IκBα, p-ERK, ERK, and CD40. FIG. 6C shows extracts from cells treated with BAFF that were analyzed with antibodies against p-IκBα, IκBα, p-ERK, ERK and tubulin. FIG. 6D shows extracts from cells treated with anti-IgM that were analyzed with antibodies against p-ERK and ERK. FIG. 6E shows extracts from B cells treated with mApril for different time that were analyzed with antibodies against p-Iκ-Bα and Actin. α-tubulin and Actin were used as a loading control. The levels for p52, p-IκBα, p-ERK and p-JNK were analyzed by Scion Image 1.62C alias and presented as relative fold of induction of the untreated samples.

FIGS. 6F-6G show increased CD40 and BAFF signaling in Act1-deficient MEFs. FIG. 6F shows wild-type and Act1-deficient MEFs that were co-transfected with flag-tagged CD40 and BAFFR with E-selectin luciferase reporter construct. The transfected cells were untreated or stimulated with CD40L or mBAFF, followed by luciferase reporter assay. The data are presented as fold of induction of luciferase activity in the treated cells relative to untreated cells. The experiments were repeated four times. The expression levels of the transfected flag-CD40 and flag-BAFFR in wild-type and Act1-deficient MEFs were measured by Western analysis with anti-flag (M2). FIG. 6G shows cell extracts were prepared from wildtype and Act1-deficient MEFs that were untreated or treated with TNFα, followed by Western analysis with antibodies against p-IκBα and p-JNK. α-tubulin was used as a loading control. The levels for p52, p-IκBα, p-ERK and p-JNK were analyzed by Scion Image 1.62C alias and presented as relative fold of induction of the untreated samples.

FIGS. 7A-7C show CD40L- and BAFF-induced interaction of Act1 with CD40, BAFFR and TRAF3. Cell extracts from IM9 (FIGS. 7A, 7B) or primary human splenocytes (FIG. 7C) untreated or treated with CD40L (FIG. 7A) or BAFF (FIGS. 7B and 7C) for the indicated times were immunoprecipitated with anti-Act1, followed by western analyses with antibodies against CD40 (FIG. 7A), BAFFR (FIGS. 7B, 7C) Act1 (FIGS. 7A, 7B and 7C), TRAF3 (FIGS. 7A, 7B), TRAF2 (FIGS. 7A, 7B) and TRAF6 (FIGS. 7A, 7B).

FIGS. 7D and 7E show Act1 functions as a negative regulator in cultured cells. FIG. 7D shows Hela cells that were transiently transfected with synthetic RNA duplex for human Act1 (5′-AAGCTCACTCAACCCTGAAACTT-3′) (SEQ ID NO: 1). 48 hr after transfection, cells were untreated or treated with CD40L for different time points. The cell extracts were subjected to western blotting with the antibodies of p-JNK, total JNK and α-tubulin (loading control). FIG. 7E shows 293 cells that were transiently co-transfected with Act1 or IRAK1 together with pE-selectin luciferase reporter and increasing amounts of TRAF3. 48 hr after transfection, cells were harvested for luciferase reporter assay. Data were shown as relative fold of induction.

FIG. 8 is a bar graph of TD responses in double knockout mice of Act1 and CD40.

FIG. 9 are bar graphs of TI responses in double knockout mice of Act1 and CD40.

FIG. 10 is a bar graph of TD responses in double knockout mice of Act1 and BAFF.

FIG. 11 are bar graphs of TI responses in double knockout mice of Act1 and BAFF.

FIG. 12 are graphs showing Sjögren syndrome like auto-antibodies in Act1-deficient mice.

FIG. 13 is a survival curve of the combined effects of Sjögren and SLE auto-immune diseases in wild type and Act1-deficient mice.

FIGS. 14A-14C are photographs of Act1-deficient mice and wild type mice which show that the Act1-deficient mice have difficulties in maintaining fully opened eyelids around 3 months of age.

FIG. 15 shows the nucleotide (SEQ ID NO: 5) and amino acid (SEQ ID NO: 6) sequences of human Act1.

FIG. 16 shows the nucleotide (SEQ ID NO: 7) and amino acid (SEQ ID NO: 8) sequences of murine Act1.

FIG. 17 is an haematoxylin and eosin (H/E) stain of the lung of an Act1-deficient mouse showing lung adenoma in Act1 deficient mice but not in wild type mice. The lung adenoma was observed by H/E staining. The pictures are shown at different amplifications (20×, 100×, 400×).

FIG. 18 is a photograph of an Act1 deficient mouse and an H/E stain of the skin of the Act1-deficient mouse showing skin fibroepithelioma in Act1 deficient mice but not in wild type mice. The skin cancer was detected by overall appearance or by H/E staining. The pictures are shown at different amplifications (100×, 400×).

FIGS. 19A-19C are bar graphs showing that Act1 is required for IL-17 mediated proinflammatory cytokine production in mouse embryo fibroblasts (MEF). FIG. 19A shows an ELISA of cytokine production. Primary MEFs isolated from Act1 wild-type and Act1 deficient mice were stimulated with IL-1 (10 ng/ml), TNF (10 ng/ml), IL-17 (50 ng/ml) or TNF (10 ng/ml) plus IL-17 (50 ng/ml) for 8 h and the cytokine levels in the culture supernatant were measured by ELISA. Act1 deficient MEFs were transfected with either empty vector or Act1 expression construct.

FIGS. 20A-20C show IL-17 mediated proinflammatory cytokine production and inflammatory responses are impaired in Act1-deficient mice. FIG. 20A is a bar graph of the results of experiments in which 8-week-old Act1-deficient mice (−/−) and Act1 wild-type (+/+) mice were intraperitoneally injected by indicated cytokines and the serum KC levels were measured by ELISA after 4 h. FIGS. 20B and 20C show the results of experiments in which 8-week-old epithelia-specific knockout mice (K18cre+/−Act1−/flox) and control mice K18cre+/−Act1+/flox were challenged intranasally with PBS or IL-17. The bronchoalveolar lavage and the lungs were collected after 24 h for KC analysis and histological staining. Data are representative of two independent experiments.

FIGS. 21A-21B show Act1 is required for IL-17 mediated IκB degradation. FIG. 21A shows primary MEFs isolated from Act1 wild-type and Act1 deficient mice were treated with 50 ng/ml IL-17 for the indicated times and analyzed by Western with antibody against IκB. FIG. 21B shows primary MEFs isolated from Act1 wild-type and Act1 deficient mice were treated with 50 ng/ml IL-17 for the indicated times and analyzed by Western with antibody against phospho-JNK and phospho-ERK.

FIG. 22 shows IL-17 receptor interacts with Act1 through it's SEF domain. 293 cells were transiently transfected with FLAG-tagged IL-17 receptor or SEF domain deletion mutant (ΔSef) and HA-tagged Act1, followed by immunoprecipation with anti-FLAG antibody (M2) and Western analysis with antibodies against Act1, TAK1, Traf3 and Traf6.

FIG. 23 is a graph showing experimental autoimmune encephalomyelitis (EAE) incidence is greatly reduced in Act1 deficient mice. EAE was induced by MOG immunization as described in Example 10. Mean clinical scores were calculated for each day for Act1 wild-type (n=5) and Act1 deficient mice (n=5).

### DETAILED DESCRIPTION OF THE INVENTION

TNF receptor (TNFR) superfamily members, CD40 and BAFFR play critical roles in B cell survival and differentiation. The present invention is based, in part, on the discovery that a genetic deficiency in a novel adapter molecule, Act1, for CD40 and BAFF results in a dramatic increase in peripheral B cells, which culminates in lymphadenopathy and splenomegaly, hypergammaglobulinemia, hyper humoral immune responses and production of autoantibodies. While the B cell specific Act1 knockout mice displayed similar phenotype, the pathology of the Act1-deficient mice was blocked in CD40-Act1 and BAFF-Act1 double knockout mice. CD40- and BAFF-mediated survival is significantly increased in Act1-deficient B cells, with stronger IκB phosphorylation, processing of NF-κB2 (p100/p52), and activation of JNK, ERK and p38 pathways, indicating that Act1 negatively regulates CD40- and BAFF-mediated signaling events. These findings demonstrate that Act1 plays an important role in the homeostasis of B cells by attenuating CD40 and BAFFR signaling. Act1 has also been shown herein to be a key component in the IL-17 signaling pathway. In addition, the Act1 knockout mice display systemic lupus erythematosus (SLE), Sjögren's syndrome and cancer (e.g., lung adenoma, skin fibroepithelioma).

The CD40 (CD40L/CD1154)-CD40 ligand and BAFF-BAFF receptor (BAFFR) cytokine systems, members of the tumor necrosis factor (TNF) superfamily, play critical roles in the homeostatic regulation of B cell functions (Schonbeck, U. and Libby, P., Cell Mol. Life Sci. 58:4-43 (2002); van Kooten, C. and Banchereau, J., Int. Arch. Allergy Immunol. 113:393-399 (1997); Bishop, G. A., et al., Immunol. Res. 24:97-109 (2001); Lei, X. F., et al., J. Clin. Invest 101:1342-1353 (1998); Thompson, J. S., et al., Science 293:2108-2111 (2001); Mackay, F. and Browning, J. L., Nat. Rev. Immunol. 2:465-475 (2002); Mackay, F., et al., Annu. Rev. Immunol. 21:231-264 (2003)). The CD40-mediated pathway has been shown to play important roles in T cell-mediated B lymphocyte activation. Ligation of B cell CD40 by CD40L (CD154) expressed on activated T cells stimulates B cell survival, proliferation, differentiation, isotype switching, upregulation of surface molecules contributing to antigen presentation, development of the germinal center (GC), and the memory B cell response. Mice deficient in expression of CD40L or CD40 were unable to mount a primary or a secondary antibody response to a T cell-dependent antigen, did not form GCs, and did not generate antigen-specific memory B cells (Kawabe, T., et al., Immunity 1: 167-178 (1994); Xu, J., et al., Immunity 1:423-431 (1994)).

BAFF (also known as BlyS, TALL-1, zTNF4, THANK, and TNFSF 13B), a recently defined member of the TNF family has emerged as an important regulator of B cell homeostasis. In BAFF-deficient mice, peripheral B cell survival is severely perturbed, resulting in a complete loss of follicular and marginal zone B lymphocytes (Mackay, F. and Browning, J. L., Nat. Rev. Immunol. 2:465-475 (2002); Thompson, J. S., et al., Science 293:2108-2111 (2001); Schiemann, B., et al., Science, 293:2111-2114 (2001); Shu, H. B., et al., J. Leukoc. Biol. 65:680-683 (1999); Khare, S. D., et al., Proc. Natl. Acad. Sci. USA 97:3370-3375 (2000)). Conversely, mice overexpressing BAFF (Blys/TALL-1) display mature B cell hyperplasia and symptoms of systemic lupus erythematosus (SLE) (Gross, J. A., et al., Nature 404:995-999 (2000)). BAFF (Blys/TALL-1) exerts its effect by binding three receptors: transmembrane activator of and CAML interactor (TACI), B cell maturation antigen (BCMA), and BAFF receptor (BAFFR/BR3) (Thompson, J. S., et al., Science 293:2108-2111 (2001); Schiemann, B., et al., Science, 293:2111-2114 (2001); Mackay, F. and Browning, J. L., Nat. Rev. Immunol. 2:465-475 (2002); Mackay, F., et al., Annu. Rev. Immunol. 21:231-264 (2003)). In BCMA-deficient mice, no gross effect on B cell development or antigen-specific immune responses has been observed. In contrast, mature B cells accumulate in TACI-deficient mice, suggesting that TACI may negatively regulate B cell survival and development (Yan, M., et al., Nat. Immunol. 2:638-643 (2001); Seshasayee, D., et al., Immunity 18:279-288 (2003)). Lastly, mice expressing a naturally mutated form of BAFFR (the A/WySnJ strain) exhibit peripheral B cell abnormalities comparable to those found in BAFF(Blys/TALL-1)-deficient mice, indicating that BAFF-BAFFR interaction is primarily responsible for peripheral B cell survival and development (Thompson, J. S., et al., Science 293:2108-2111 (2001)).

CD40 and BAFFR ligation activate the NFκB family of transcription factors, which are critical for the regulation of B cell survival and development. NFκB transcription factors are homo- or heterodimers of a group of structurally related proteins, including Rel (c-Rel), RelA (p65), RelB, NFκB1 (p50 and its precursor p105), and NFκB2 (p52 and its precursor p100) (Ghosh, S. and Karin, M., Cell 109 Suppl, S81-S96 (2002)). In resting cells, most NFκB/Rel dimers are bound to IκBs and retained in the cytoplasm. Upon stimulation with CD40L and BAFF, the IκB proteins are phosphorylated by IκB kinase (IKK) (DiDonato, J. A., et al., Nature 388: 548-554; Mercurio, F., et al., Science 278:860-866 (1997); Regnier, C. H., et al., Cell 90:373-383 (1997); Woronicz, J. D., et al., Science 278:866-869 (1997); Zandi, E., et al., Cell 91:243-252 (1997)). IKK is composed of the catalytic subunits IKKα and IKKβ and the regulatory subunit IKKγ. It has been shown that IKKβ, but not IKKα is mainly responsible for the IκB phosphorylation, which is followed by rapid ubiquitination and degradation of the IκB proteins (Karin, M. and Ben Neriah, Y., Annu. Rev. Immunol. 18:621-663 (2000); Ghosh, S., et al., Annu. Rev. Immunol. 16, 225-260 (1998)), releasing NFκB (p65/p50) to activate transcription in the nucleus (the canonical NFκB activation pathway). Conditional deletion of IKKβ results in the rapid loss of B cells (Li, Z. W., et al., J. Immunol. 170: 4630-4637 (2003); Pasparakis, M., et al., J. Exp. Med. 196:743-752 (2002)), indicating that the canonical NFκB activation pathway mediated by IKKβ is probably required for the general differentiation and homeostasis of B cells. CD40 and BAFFR are also able to induce a noncanonical NFκB2 processing pathway, that is, IKKαleads to the phosphorylation and processing of p100, resulting in the formation of an active RelB/p52 heterodimer (Coope, H. J., et al. EMBO J. 21:5375-5385 (2002); Kayagaki, N., et al., Immunity 17:515-524 (2002)). NFκB inducing kinase (NIK) was shown to play a role in activating IKKα, leading to p 100 processing (Ninomiya-Tsuji, J., et al., Nature 398:252-256 (1999); Irie, T., et al., FEBS Lett. 467:160-164 (2000); Takaesu, G., et al., Mol. Cell 5:649-658 (2000); Takaesu, G., et al., Mol. Cell Biol. 21:2475-2484(2001); Qian, Y., et al., J. Biol. Chem. 276: 41661-41667 (2001); Deng, L., et al., Cell 103, 351-361 (2000); Wang, C., et al., Nature 412:346-351 (2001); Yin, L., et al., Science 291:2162-2165 (2001); Garceau, N., et al., J. Exp. Med. 191, 381-386 (2000)). The fact that mature B cell numbers are reduced in mice lacking NIK or p52, and in irradiated mice reconstituted with IKKα-deficient lymphocytes suggests the important role of this noncanonical NFκB activation pathway in CD40 and BAFFR-mediated B cell survival.

CD40 and BAFFR utilize TRAF (TNF receptor-associated factor) molecules as receptor-proximal adapters to mediate the activation of downstream kinases including IKKs and MAP kinases (Xu, L. G. and Shu, H. B., J. Immunol. 169:6883-6889 (2002); Hostager, B. S. and Bishop, G. A., J. Immunol. 162, 6307-6311 (1999)). As described herein, a novel adapter molecule, called Act1, has been identified as an important regulator in signaling pathways mediated by CD40 and BAFF. Endogenous Act1 is recruited to CD40 in B cells upon stimulation with CD40 ligand (CD40L). Act1 does not have any enzymatic domains; instead it contains a helix-loop-helix at the N-terminus and a coiled-coil at the C-terminus (Qian, Y., et al., Proc. Natl. Acad. Sci. USA 99:9386-9391 (2002); Li, X., et al., Proc. Natl. Acad. Sci. USA 97:10489-10493 (2000); Leonardi, A., et al., Proc. Natl. Acad. Sci. USA 97:10494-10499 (2000)). In addition, Act1 contains two putative TRAF binding sites, EEESE (residues 38-42) (SEQ ID NO: 2) and EERPA (residues 333-337) (SEQ ID NO: 3) (Qian, Y., et al., Proc. Natl. Acad. Sci. USA 99:9386-9391 (2002)). To elucidate the physiological functions of Act1, Act1-deficient mice were generated. Act1-deficient mice revealed major lymphoid system defects, which is marked with lymphadenopathy, hypergammaglobulinemia and production of autoantibodies. A general increase in the numbers of peripheral B cells, and CD40- and BAFFR-mediated B cell survival is significantly increased in Act1-deficient mice, indicating that Act1 is an important modulator in humoral immune responses by regulating CD40 and BAFFR signaling in B cells. In addition, Act1 deficient mice revealed that Act1 is a key component in the IL-17 signaling pathway.

Accordingly, the present invention also provides a transgenic non-human mammal which lacks a functional Act1 gene referred to herein as a “transgenic non-human Act1 knockout mammal” or a “Act1 knockout mammal”. In a particular embodiment, the genome of the Act1 knockout mammal comprises at least one non-functional allele for the endogenous Act1 gene. Thus, the invention provides a source of cells (for example, tissue, cells, cellular extracts, organelles) and transgenic non-human mammals useful for studying Act1. Further aspects of the invention provide methods for producing transgenic non-human Act1 knockout mammals and transgenic non-human Act1 deficient mammals produced by the methods; targeting vectors for use in producing Act1 deficient mammals; methods for the identification of agents (for example, diagnostic or therapeutic agents) which inhibit or mimic Act1 activity; and methods of identifying agents that can be used to treat and/or prevent a disease or condition associated with a defect in Act1 (e.g., systemic lupus erythematosus (SLE); Sjögren's syndrome).

Any suitable non-human mammal can be used to produce the Act1 knockout mammal described herein. For example, a suitable mammal can be a rodent, a canine, a feline, an ovine, a bovine, a porcine and a caprine. Specific examples of suitable mammals include a mouse, a rat, a dog, a cat, a sheep, a cow, a pig, a goat and a rabbit.

As used herein, the term “gene” refers to DNA sequences which encode the genetic information (for example, nucleic acid sequence) required for the synthesis of a single protein (for example, polypeptide chain). The term “Act1 gene” refers to a particular mammalian gene which comprises a DNA sequence which encodes Act1. An “allele” is an alternative from of gene found at the same locus of a homologous chromosome. Homologous chromosomes are chromosomes which pair during meiosis and contain identical loci. The term locus connotes the site (for example, location) of a gene on a chromosome.

As used herein the terms “transgenic non-human Act1 knockout mammal” and “Act1 knockout mammal” refer to a mammal whose genome comprises a disrupted or inactivated Act1 gene. Those of skill in the art will recognize that the term “knockout” refers to the functional inactivation (knockdown) of the gene. The disruption introduces a chromosomal defect (for example, mutation or alteration) in the Act1 gene at a point in the nucleic acid sequence that is important to either the expression of the Act1 gene or the production of a functional Act1 protein (for example, polypeptide). The disruption can also introduce a chromosomal defect in a region other than the Act1 gene wherein the disruption results in an inactivated Act1 gene. Thus, the introduction of the disruption inactivates the endogenous target gene (for example, Act1 gene).

As used herein the terms “disruption”, “functional inactivation”, “knockout”, knockdown” “alteration” and “defect” connote a partial or complete reduction in the expression and/or function of the Act1 polypeptide encoded by the endogenous gene of a single type of cell, selected cells (for example, B cells) or all of the cells of a non-human transgenic Act1 knockout animal. Thus, according to the instant invention the expression or function of the Act1 gene product can be completely or partially disrupted or reduced (for example, by about 50%, 75%, 80%, 90%, 95% or more) in a selected group of cells (for example, a tissue or organ) or in the entire animal. As used herein, the term “a functionally disrupted Act1 gene” includes a modified genome wherein the modification in the genome results in failure of expression of Act1 polypeptide (partially such as low levels of expression, completely such as lack of expression) or expression of a nonfunctional (partially, completely) Act1 protein; and a modified Act1 gene which fails to express an Act1 polypeptide or which expresses an Act1 polypeptide that lacks completely or partially the biologically activity of Act1 (e.g., a truncated polypeptide having less than the entire amino acid polypeptide chain of a wild-type Act1 polypeptide and is partially or completely non-functional; a mutated Act1 polypeptide which is partially or completely non-functional).

Disruption of the Act1 gene can be accomplished by a variety of methods known to those of skill in the art. For example, gene targeting using homologous recombination, mutagenesis (for example, point mutation), RNA interference (e.g., small interfering RNA (siRNA), short hairpin RNA (shRNA)) and anti-sense technology can be used to disrupt an Act1 gene.

In a particular embodiment, the invention provides a transgenic knockout mammal whose genome comprises either a homozygous or heterozygous disruption of its Act1 gene. A knockout mammal whose genome comprises a homozygous disruption is characterized by somatic and germ cells which contain two nonfunctional (disrupted) alleles of the Act1 gene while a knockout mutant whose genome comprises a heterologous disruption is characterized by somatic and germ cells which contain one wild-type allele and one nonfunctional allele of the Act1 gene.

As used herein, the term “genotype” refers to the genetic makeup of an animal with respect to the Act1 chromosomal locus. More specifically the term genotype refers to the status of the animal's Act1 alleles, which can either be intact (for example, wild-type or +/+); or disrupted (for example, knockout) in a manner which confers either a heterozygous (for example, +/−); or homozygous (−/−) knockout genotype.

The present invention also provides methods of producing a transgenic non-human mammal which lacks a functional Act1 gene. Briefly, the standard methodology for producing a transgenic embryo requires introducing a targeting construct, which is designed to integrate by homologous recombination with the endogenous nucleic acid sequence of the targeted gene, into a suitable ES cells. The ES cells are then cultured under conditions effective for homologous recombination between the recombinant nucleic acid sequence of the targeting construct and the genomic nucleic acid sequence of the host cell chromosome. Genetically engineered stem cell that are identified as comprising a knockout genotype which comprises the recombinant allele is introduced into an animal, or ancestor thereof, at an embryonic stage using standard techniques which are well known in the art (for example, by microinjecting the genetically engineered ES cell into a blastocyst). The resulting chimeric blastocyst is then placed within the uterus of a pseudo-pregnant foster mother for the development into viable pups. The resulting viable pups include potentially chimeric founder animals whose somatic and germline tissue comprise a mixture of cells derived from the genetically-engineered ES cells and the recipient blastocyst. The contribution of the genetically altered stem cell to the germline of the resulting chimeric mice allows the altered ES cell genome which comprises the disrupted target gene to be transmitted to the progeny of these founder animals thereby facilitating the production of transgenic “knockout animals” whose genomes comprise a gene which has been genetically engineered to comprise a particular defect in a target gene.

In a particular embodiment of the present invention, a transgenic Act1 knockout mammal is produced by introducing a targeting vector which disrupts the Act1 gene into an ES cell thereby producing a transgenic stem cell. A transgenic ES cell which includes the disrupted Act1 gene due to the integration of the targeting vector into its genome is selected and introduced into a blastocyst, thereby forming a chimeric blastocyst. The chimeric blastocyst is introduced into the uterus of a pseudo-pregnant mammal wherein the pseudo-pregnant mammal gives birth to a transgenic non-human mammal which lacks a functional Act1 gene.

As a result of the disruption of the Act1 gene, the Act1 knockout mammal of the present invention can manifest a particular phenotype. The term phenotype refers to the resulting biochemical or physiological consequences attributed to a particular genotype. In one embodiment, the Act1 knockout mammal displays lymphoid system abnormalities (e.g., enlarged lymph nodes (e.g., cervical, axillary, brachial), lymphoid hyperplasia, increased germinal centers, accumulation of large numbers of immunoglobulin-producing plasma cells (Syndecan-1 positive cells) in the medulla of the lymph nodes); hypergammaglobulinemia; production of autoantibodies and combinations thereof. The phenotype can further comprise inflammation of tissue (e.g., upper respiratory airway, skin). The Act1 knockout mammal can also display SLE and Sjögren's syndrome. In addition, the phenotype of the Act1 knockout non-human cell or mammal can include the development of cancer (e.g., lung adenoma, skin fibroepithelioma).

One of skill in the art will easily recognize that the Act1 gene can be disrupted in a number of different ways, any one of which may be used to produce the Act1 knockout mammals of the present invention. For example, a transgenic knockout animal according to the instant invention can be produced by the method of gene targeting. As used herein the term “gene targeting” refers to a type of homologous recombination which occurs as a consequence of the introduction of a targeting construct (for example, vector) into a mammalian cell (for example, an ES cell) which is designed to locate and recombine with a corresponding portion of the nucleic acid sequence of the genomic locus targeted for alteration (for example, disruption) thereby introducing an exogenous recombinant nucleic acid sequence capable of conferring a planned alteration to the endogenous gene. Thus, homologous recombination is a process (for example, method) by which a particular DNA sequence can by replaced by an exogenous genetically engineered sequence. More specifically, regions of the targeting vector which have been genetically engineered to be homologous (for example, complementary) to the endogenous nucleotide sequence of the gene which is targeted for disruption line up or recombine with each other such that the nucleotide sequence of the targeting vector is incorporated into (for example, integrates with) the corresponding position of the endogenous gene.

One embodiment of the present invention provides a vector construct (for example, an Act1 targeting vector or Act1 targeting construct) designed to disrupt the function of a wild-type (endogenous) mammalian Act1 gene. In a particular embodiment, an effective Act1 targeting vector comprises a recombinant sequence that is effective for homologous recombination with the Act1 gene. For example, a replacement targeting vector comprising a genomic nucleotide sequence which is homologous to the target sequence operably linked to a second nucleotide sequence which encodes a selectable marker gene exemplifies an effective targeting vector. Integration of the targeting sequence into the chromosomal DNA of the host cell (for example, an ES cell) as a result of homologous recombination introduces an intentional disruption, defect or alteration (for example, insertion, deletion) into the sequence of the endogenous gene. One aspect of the present invention is to delete, replace (e.g., mutate) all or part of the nucleotide sequence of a non-human mammalian gene which encodes the Act1 polypeptide. In one embodiment, an (one or more) exon(s) of the Act1 gene is disrupted (e.g., exon 1, exon 2, exon 3, exon 4, exon 5, exon 6, exon 7, exon 8, exon 9). In another embodiment, a portion of an exon (or portions of one or more exons) is disrupted. In a particular embodiment, a segment which includes exon 2 of the Act1 gene, is removed.

One of skill in the art will recognize that any Act1 genomic nucleotide sequence of appropriate length and composition to facilitate homologous recombination at a specific site that has been preselected for disruption can be employed to construct a Act1 targeting vector. Guidelines for the selection and use of sequences are described for example in Deng and Cappecchi, Mol. Cell. Biol., 12:3365-3371 (1992) and Bollag, et al., Annu. Rev. Genet., 23:199-225 (1989). For example, a wild-type Act1 gene can be mutated and/or disrupted by inserting a recombinant nucleic acid sequence (for example, a Act1 targeting construct or vector) into all or a portion of the Act1 gene locus. For example, a targeting construct can be designed to recombine with a particular portion within the enhancer, promoter, coding region, start codon, non-coding sequence, introns or exons of the Act1 gene. Alternatively, a targeting construct can comprise a recombinant nucleic acid which is designed to introduce a stop codon after one or more exons of the Act1 gene. In a particular embodiment, an Act1 gene targeting construct comprises a 5′ arm comprising the first intron of the Act1 gene, followed by a first recombination site (e.g., loxP site), followed by a marker gene (e.g., Neo gene), followed by a recombination site, followed by one or more exons of the Act1 gene (e.g., exon 2), followed by a recombination site, followed by a 3′ arm comprising a fragment from the second intron of the Act1 gene.

Suitable targeting constructs of the invention can be prepared using standard molecular biology techniques known to those of skill in the art. For example, techniques useful for the preparation of suitable vectors are described by Maniatis, et al., Molecular Cloning: A Laboratory Manual, Cold Spring Harbor Laboratory, Cold Spring Harbor, N.Y. Appropriate vectors include a replacement vector such as the insertion vector described by Capecchi, M. R., Science, 244:1288-92 (1989); or a vector based on a promoter trap strategy or a polyadenylation trap, or “tag-and-exchange” strategy as described by Bradley, et al., Biotechnology, 10:543-539 (1992); and Askew, et al., Mol. Cell. Biol., 13:4115-5124 (1993).

One of skill in the art will readily recognize that a large number of appropriate vectors known in the art can be used as the basis of a suitable targeting vector. In practice, any vector that is capable of accommodating the recombinant nucleic acid sequence required to direct homologous recombination and to disrupt the target gene can be used. For example, pBR322, pACY164, pKK223-3, pUC8, pKG, pUC19, pLG339, pR290, pKC11 or other plasmid vectors can be used. Alternatively, a viral vector such as the lambda gt11 vector system can provide the backbone (for example, cassette) for the targeting construct.

According to techniques well known to those of skill in the art, genetically engineered (for example, transfected using electroporation or transformed by infection) ES cells, are routinely employed for the production of transgenic non-human embryos. ES cells are pluripotent cells isolated from the inner cell mass of mammalian blastocyst. ES cells can be cultured in vitro under appropriate culture conditions in an undifferentiated state and retain the ability to resume normal in vivo development as a result of being combined with blastocyst and introduced into the uterus of a pseudo-pregnant foster mother. Those of skill in the art will recognize that various stem cells are known in the art, for example AB-1, HM-1, D3. CC1.2, E-14T62a, RW4 or JI (Teratomacarcinoma and Embryonic Stem Cells: A Practical Approach, E. J. Roberston, ed., IRL Press).

The present invention also provides methods of producing a transgenic non-human mammal which is a B cell specific Act1 deficient mammal (B cell specific Act1 knockout (Act1-deficient) non-human mammal). In one embodiment, the B cell specific Act1 knockout non-human mammal can be produced by breeding an Act1 deficient non-human mammal with a CD19cre non-human mammal to generate CD19cre^{+/-}Act1^{+/-} non-human mammals. These non-human mammals can be further bred onto Act1 floxed non-human mammals to generate control transgenic non-human mammals and B cell specific knockout non-human mammals.

It is to be understood that the Act1 knockout mammals described herein can be produced by methods other than the ES cell method described above, for example by the pronuclear injection of recombinant genes into the pronuclei of one-cell embryos or other gene targeting methods which do not rely on the use of a transfected ES cell, and that the exemplification of the single method outlined above is not intended to limit the scope of the invention to animals produced solely by this protocol.

The transgenic Act1 knockout mammals described herein can also be bred (for example, inbred, outbred or crossbred) with appropriate mates to produce colonies of animals whose genomes comprise at least one non-functional allele of the endogenous gene which naturally encodes and expresses functional Act1. Examples of such breeding strategies include but are not limited to: crossing of heterozygous knockout animals to produce homozygous animals; outbreeding of founder animals (for example, heterozygous or homozygous knockouts), or with a non-human mammal, such as a mouse, whose inbred genetic background has been altered.

In particular embodiments, the transgenic Act1 knockout mammal can be bred onto a CD40 knockout non-human mammal (CD40^{-/-}) or a BAFF knockout mammal (BAFF^{-/-}) to first generate heterozygous mice (Act1^{+/-}CD40^{+/-} or Act1^{+/-}BAFF^{+/-}), then a CD40-null Act1 heterozygous mammal (CD40^{-/-}Act1^{+/-}) or a BAFF-null Act1 heterozygous mammal (BAFF^{-/-} Act1^{+/-}). These mammals can then be bred among themselves to generate CD40-Act1 (CD40^{-/-}Act1^{+/-}) and BAFF-Act 1 (BAFF-Act1^{-/-}) double knockout mice.

In another embodiment, transgenic Act1 knockout mice can be bred with the XenoMouse® (Abgenix) to produce progeny mice capable of generating high affinity human antibodies for diagnosis or therapy.

The present invention is also directed to transgenic non-human mammals produced using the methods described herein.

In an alternative embodiment of the instant invention, transgenic ES cells can be engineered to comprise a genome which comprises disruptions of more than one gene whose polypeptide product has been implicated in Act1 signaling (E. G., Traf molecules such as the Traf3 molecule). Transgenic non-human Act1 deficient non-human mammals can be produced using such ES cells as described herein.

The Act1 knockout mammals, cell lines, primary tissue or cell cultures, cellular extracts or cell organelles isolated from the Act1 knockout mammals of the instant invention are useful for a variety of purposes. In one embodiment of the present invention the transgenic Act1 knockout mammals produced in accordance with the present invention are utilized as a source of cells for the establishment of cultures or cell lines (for example, primary, or immortalized), which are useful for the elucidation of the roles in Act1 in cellular function. Such cells, which can be isolated from mammalian tissues, include lymphocytes, for example, B cells. The primary cell cultures, or cell lines, can be derived from any desired tissue or cell-type which normally express high levels of Act1 mRNA, including but not limited to B cells.

For example, it is desirable to produce panels of cell lines which differ in their expression of one of more genes. Thus, the present invention encompasses a cell line in which an endogenous Act1 gene has been disrupted (for example, Act1 knockout cells or cell lines such as a B cell line or an epithelial cell line). The resulting Act1-functionally disrupted cell comprises a genotype which differs from its parental wild-type cell in a defined manner and thereby allows for the elucidation of the effects of Act1-deficiency on antigen (e.g., TD antigen, TI antigen) stimulation, particularly in B cells. In another embodiment, an Act1 knockout cell or cell line can be engineered using skills known in the art. For example, cells which do not possess an endogenous Act1 gene or which normally do not express Act1 can be engineered to do so. For example, an exogenous Act1 gene can be introduced into a cell which does not possess an endogenous Act1 gene wherein the cell expresses Act1 due to the presence of the exogenous Act1 gene. Alternatively, exogenous nucleic acid can be spliced into the genome of a cell which does not normally express Act1 in order to “turn on” the normally silent Act1 gene. The agent can be for example, a nucleic acid molecule, a polypeptide, an organic molecule, an inorganic molecule, a fusion protein etc. silent, endogenous Act1 gene. Subsequently the Act1 gene in the engineered cells can be disrupted using the methods described herein and known to those of skill in the art for use in the methods and compositions of the present invention.

The availability of Act1 knockout cells and mammals (for example, homozygous, heterozygous) facilitate the genetic dissection of Act1-mediated signaling pathways and allow for the identification of Act1 specific enhancers and/or inhibitors. For example, an agent which modulates (inhibits/enhances) one or more functions of Act1 equally in a knockout cell line and its wild-type parental cell line would be recognized as a non-Act1-specific modulator (e.g, inhibitor, enhancer), while an agent which modulates an Act1-dependent function in a wild-type cell line which has no effect in the knockout cell line would be recognized as an Act1-specific modulator. Further, the use of cell lines and Act1 knockout mammals which have one or more disruptions in the Act1 gene facilitate the identification of agents with potential therapeutic value for the treatment of diseases in which altered Act1 function plays a role (e.g., SLE, Sjögren's syndrome).

The invention provides methods (e.g., in vivo, in vitro) of identifying an agent that modulates mammalian Act1 (for example, an antagonist, an agonist, a partial antagonist or agonist). A modulator of Act1 includes any agent that modulates Act1 gene expression (partial or complete) or function (partial or complete) of the Act1 protein. According to the instant invention, the agent can be combined with a cell (for example, B cells, epithelial cells), a tissue, a cellular extract or organelle, and/or administered to a whole animal. As demonstrated in the following examples, combining and/or administering an agent can be accomplished in various ways such as the addition to culture media, tissue perfusion, by expressing it from a vector, or by injection.

In one embodiment of the present invention an in vitro screening method for determining whether an agent inhibits Act1 is provided. In one embodiment, the in vitro screening method can comprise contacting a cell which comprise a wild type Act1 gene (e.g., B cell; epithelial cell) with an agent to be assessed. The phenotype of the cell to which the agent has been contacted is assessed and compared to the phenotype of the Act1 knockout cell (e.g., assessing whether the cell develops a cancerous phenotype using HE staining). If the phenotype of the cell is the about same when compared to the phenotype of the knockout cell, then the agent inhibits Act1. In a particular embodiment, the agent to be assessed can also be contacted with a cell that lacks a functional Act1 gene and the phenotypes of the cells can be compared.

In vivo methods of identifying an agent that modulates mammalian Act1 can be used. In one embodiment of the present invention an in vivo screening method for determining whether an agent inhibits Act1 is provided. In one embodiment, the in vivo screening method can comprise administering to a non-human mammal which comprise a wild type Act1 gene (wild-type mammal), an agent to be assessed. The phenotype of the wild type mammal to whom the agent has been administered is assessed and compared to the phenotype of the Act1 knockout mammal. If the phenotype of the wild type mammal is the about same when compared to the phenotype of the knockout mammal, then the agent inhibits Act1. In a particular embodiment, the agent to be assessed can also be administered to a transgenic non-human mammal which lacks a functional Act1 gene (Act1 knockout mammal) and the phenotypes of the wild type and the Act1 knockout mammal are then compared. In addition, the method can further comprise administering to the mammals an amount of antigen sufficient to stimulate an immune response and comparing the phenotypes of the mammals in the presence of the agent and the antigen.

The invention also provides a method of identifying an Act1 mimic (an agent that exhibits (mimics) Act1 activity, such as a recombinant peptide, polypeptide, fusion protein or small molecule). According to this embodiment of the invention, the transgenic Act1 knockout mammals or their isolated cells, tissues, cellular extracts or organelles provide a starting material, or control material, in which the function of potential Act1 mimics can be evaluated. In one embodiment, the method comprises introducing the agent into cells which lack a functional Act1 gene and determining whether a Act1-mediated cellular function (one or more) occurs in the presence of the agent. If Act1-mediated cellular function occurs in the cells which lack a functional Act1 gene in the presence of the agent, then the agent is an Act1 mimic. In a particular embodiment, the method comprises contacting a cell which lacks a functional Act1 gene (e.g., a B cell; an epithelial cell) with an agent to be assessed. Act1 deficient cells express higher levels of anti-apoptotic genes (e.g., bcl, x1) compared to a wild type cell. Therefore, genes expressed in the cell in the presence of the agent to be assessed can be determined, wherein a decrease in the expression of antiapoptotic gene(s) indicate that the agent mimics Act 1.

In another embodiment, the method of identifying an agent which mimics Act1 activity comprises introducing the agent into a transgenic non-human mammal which lacks a functional Act1 gene and determining whether a Act 1-mediated cellular function (one or more) occurs in the presence (upon or following administration) of the agent. If Act1-mediated cellular function occurs in the transgenic non-human mammal which lacks a functional Act1 gene in the presence of the agent, then the agent is an Act1 mimic. In a particular embodiment, the method for determining whether an agent mimics Act1 comprises obtaining B cells from the bone marrow of an Act1 deficient (Act1 knockout) non-human mammal (e.g., mouse). Such B cells will have reduced B cell function compared to B cells of a wild type mammal. The B cells are contacted with an agent to be assessed, thereby producing treated cells. The treated cells are then introduced into a non-human mammal which has no B cells (e.g., an irradiated non-human, wild type mammal). If an Act1-mediated cellular function (one or more) occurs in the irradiated non-human mammal that has received the treated cells, then the agent is a mimic of Act1.

In the method of identifying an Act1 mimic, an Act1-mediated cellular function includes, for example, determining the phenotype of a wild type cell or mammal (to which has been administered or contacted with the agent to be assessed) compared to that of an Act1 knockout cell or mammal.

The screening methods described herein can further comprise the use of any suitable control known to those of skill in the art. For example, the phenotype of the wild type cell or mammal in the presence of the agent is compared to the phenotype of the wild type cell or mammal in the absence of the agent; and/or the phenotype of the Act1 knockout cell or mammal in the presence of the agent is compared to the phenotype of the Act1 knockout cell or mammal in the absence of the agent. If the phenotype the Act1 knockout cell or mammal in the presence of the agent is similar to the phenotype of the knockout cell or mammal in the absence of the agent, then the agent inhibits Act1.

In a specific embodiment, the present invention is directed to a method of identifying an agent to treat or prevent SLE comprising administering to a transgenic non-human mammal whose genome comprises a disruption of an Act1 gene an agent to be assessed. The ability of the agent to treat (e.g., alleviate the symptoms of SLE) or prevent (partially or completely) SLE in the transgenic non-human mammal is then determined. If the agent treats or prevents SLE in the transgenic non-human mammal, then the agent can be used to treat or prevent SLE.

In another embodiment, the present invention is directed to a method of identifying an agent to treat or prevent Sjögren's syndrome comprising administering to a transgenic non-human mammal whose genome comprises a disruption of an Act1 gene an agent to be assessed. The ability of the agent to treat (e.g., alleviate the symptoms of Sjögren's syndrome) or prevent (partially or completely) Sjögren's syndrome in the transgenic non-human mammal is then determined. If the agent treats or prevents Sjögren's syndrome in the transgenic non-human mammal, then the agent can be used to treat or prevent Sjögren's syndrome.

In another embodiment, the present invention is directed to a method of identifying an agent to treat or prevent cancer comprising administering to a transgenic non-human mammal whose genome comprises a disruption of an Act1 gene an agent to be assessed. The ability of the agent to treat (e.g., alleviate the symptoms of cancer) or prevent (partially or completely) cancer from occurring in the transgenic non-human mammal is then determined. If the agent treats or prevents cancer in the transgenic non-human mammal, then the agent can be used to treat or prevent cancer.

In the methods of identifying agents as described herein, the phenotype of cells and non-human mammals described herein can be determined using a variety of methods as will be recognized by one of skill in the art. For example, and without limitation, visual inspection, staining techniques (e.g., H/E staining and/or immunofluorescence staining of cells), southern blotting, western blotting, videomicroscopy, flow cytometry, immunohistochemistry, ELISA, and FACS analysis can be used. In proliferation assays, for example, the use of thymidine incorporation, BrdU uptake, and cell counting are suitable techniques, as will be appreciated by one of skill in the art.

One of skill in the art will know of appropriate techniques for the introduction and/or expression of potential agents that modulate Act1 or mimic Act1. For example, a library of nucleotide sequences (for example, cDNA sequences) encoding potential modulators or mimics could be introduced (for example, transfected or transduced in the context of an expression vector) and expressed into an appropriate host cell isolated from the knockout non-human mammals provided herein, or into a host cell which has been produced via homologous recombination using an Act1 targeting vector according to the instant invention, and screened for the modulation or restoration of an Act1-dependent cellular function. For example a potential Act1 mimic includes recombinant nucleic acid sequences which encode a truncated Act1 polypeptide in combination with a nucleic acid comprising coding sequence derived from another protein (for example a fusion protein), for example nucleic acid sequence which encodes a domain of another Act1-related protein, or nucleic acid sequence which provides for example an inducible or repressible promoter sequence or which introduces a cis-acting regulatory sequence. Thus, potential modulators or mimics can include portions of a recombinant or naturally occurring Act1 polypeptide derived from the same mammalian species or from a different mammalian species.

As described herein, Act1 deficient transgenic mice developed hypergammaglobulinemia and generated significantly increased high affinity antibodies to T-cell dependent and T-cell independent antigens compared to wild type mice. Accordingly, the present invention is directed to a method of producing antibodies which specifically bind to an antigen comprising introducing the antigen into a transgenic non-human mammal whose genome comprises a disruption of an Act1 gene under conditions in which antibodies which specifically bind to the antigen are produced in the mammal. The antibodies produced by the methods described herein can be polyclonal or monoclonal antibodies. The method can further comprise isolating the antibodies which specifically bind to the antigen from the transgenic non-human mammal. A variety of antigens known in the art can be used in the methods. For example, the antigen can be a T cell independent antigen and a T cell dependent antigen. In addition, the antigen can be one which does not initiate a strong antibody response in a wild type mammal. In a particular embodiment, the antibodies produced by the methods described herein have a higher affinity for the antigen when compared to antibodies made in a wild type non-human mammal. As discussed above, the transgenic Act1 knockout mice of the present invention can be bred with the XenoMouse® (Abgenix) to produce progeny mice capable of generating high affinity human antibodies for diagnosis or therapy. Such transgenic mice can be used in the methods of producing antibodies which specifically bind to an antigen to generate high affinity human antibodies for diagnosis or therapy.

The present invention is also directed to methods of producing a hybridoma which expresses a monoclonal antibody that is specifically directed to an antigen comprising introducing the antigen into a transgenic non-human mammal whose genome comprises a disruption of an Act1 gene, under conditions in which antibodies which specifically bind to the antigen are produced in the mammal. The B cells are then isolated from the mammal, and a B cell which expresses an antibody that specifically recognizes the antigen is selected. The selected B cell is fused with an immortal cell, thereby producing a hybridoma which expresses a monoclonal antibody that is specifically directed to the antigen. The present invention is also directed to a hybridoma produced by the methods described herein.

Methods of producing a monoclonal antibody that is specifically directed to an antigen are also encompassed by the present invention. In one embodiment, the method comprised introducing the antigen into a transgenic non-human mammal whose genome comprises a disruption of an Act1 gene, under conditions in which antibodies which specifically bind to the antigen are produced in the mammal. B cells are then isolated from the mammal, and a B cell which expresses an antibody that specifically recognizes the antigen is selected. The selected B cell is fused with an immortal cell, thereby producing a hybridoma which expresses a monoclonal antibody that is specifically directed to the antigen, and the hybridoma is maintained under conditions in which the monoclonal antibody is expressed, thereby producing a monoclonal antibody that is specifically directed to an antigen. The present invention is also directed to a monoclonal antibody produced by the methods described herein.

Methods of treatment or prevention of conditions or diseases (for example, SLE, Sjögren's syndrome, cancer) associated with aberrant Act1 function are also encompassed by the invention. For example the invention provides a method of treating (for example, completely or partially alleviating the symptoms of) or preventing (for example, in a individual who is predisposed to develop) a condition or disease associated with aberrant Act1 expression. In one embodiment the invention provides a method of increasing Act1 function in an individual comprising administering to the individual an agent which exhibits Act1 activity (e.g., enhances), or is an Act1 mimic. In a second embodiment the invention provides a method of decreasing Act1 function in an individual comprising administering to the individual an agent which inhibits Act1 activity.

The agent for use in the methods of the present invention can be for example, a nucleic acid molecule (for example, DNA, RNA, anti-sense DNA, anti-sense RNA, siRNA), a protein, a peptide, a polypeptide, a glycoprotein, a polysaccharide, an organic molecule, an inorganic molecule, a fusion protein etc.

The agents (for example, therapeutic agents such as Act1 inhibitors or Act1 mimics) can be administered to a host in a variety of ways. Potential routes of administration include intradermal, transdermal (for example, utilizing slow release polymers), intramuscular, intraperitoneal, intravenous, inhalation, subcutaneous or oral routes. Any convenient route of administration can be used, for example, infusion or bolus injection, or absorption through epithelial or mucocutaneous linings. The agent can be administered in combination with other components such as pharmaceutically acceptable excipients, carriers, vehicles or diluents.

In the treatment methods designed to inhibit the function of Act1, an “effective amount” of the agent is administered to an individual. As used herein the term “effective amount” an amount that inhibits (or reduces) the activity of Act1, and results in a significant (for example, a statistically significant) difference (for example, increase, decrease) in a cellular function which is normally subject to regulation (for example, negative regulation) by Act1. The amount of agent required to inhibit Act1 activity will vary depending on a variety of factors including the size, age, body weight, general health, sex and diet of the host as well as the time of administration, and the duration or stage of the particular condition or disease which is being treated. Effective dose ranges can be extrapolated from dose-response curves derived in vitro or an in vivo test system which utilizes the transgenic non-human Act1 mammals described herein.

### EXEMPLIFICATION

### Example 1

### Generation of Act1-Deficient Mice

Experimental Procedures

The Act1 genomic clone was obtained from screening of a 129/sv BAC library. The HindIII/BamHI genomic fragment containing the exon 2 of the Act1 gene (residues 1-268, containing the first ATG) was subcloned into the Tri-Neo vector (containing three loxP sites). While the neomycin-resistance gene (Neo) was inserted between the first and the second loxP sites, HindIII/BamHI genomic fragment containing the exon 2 of the Act1 gene was flanked by the second and the third loxP sites. The resulting DNA fragment including lox-Neo-lox-exon2-lox was then subcloned into the pBS (pBlueScript) vector. For construction of the Act1 gene-targeting vector, the 5′arm (or left arm (LA) consisting of a 3.2-kilobase (kb) fragment from the first intron of the Act1 gene) was subcloned upstream of lox-Neo-lox-exon2-lox in pBS, while the 3′ arm (or right arm (RA) consisting of a 4.0-kilobase (kb) fragment from the second intron of the Act1 gene) was subcloned downstream of lox-Neo-lox-exon2-lox in pBS. Diphtoxin A gene was inserted downstream of the 3′arm for negative selection. The resulting Act1 gene-targeting vector was then linearized with Not I, electroporated into 129/sv ES cells, followed by selection for G418-resistant ES clones. The G418-resistant ES clones were then transfected with Cre to remove the Neo drug marker and the exon 2 of Act1, followed by Southern blots with two probes (prb1 and prb2) located outside of each end of the targeting construct. Targeted ES cells (Act1-null) were injected into mouse blastocysts to generate wild-type, heterozygous and homozygous mice (BALB/c). Act1-deficient mice and their age- and gender-matched wild-type littermates from these intercrosses were used for experiments. The Cleveland Clinic Foundation Animal Research Committee approved all of the animal protocols used in this study.

Generation of B Cell Specific Act1 Deficient Mice

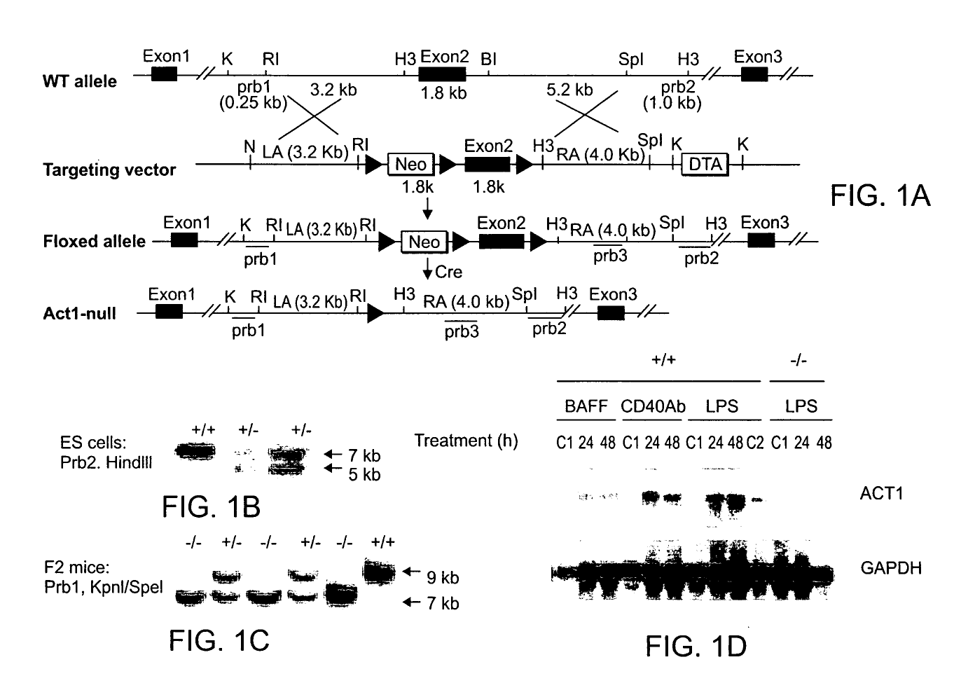

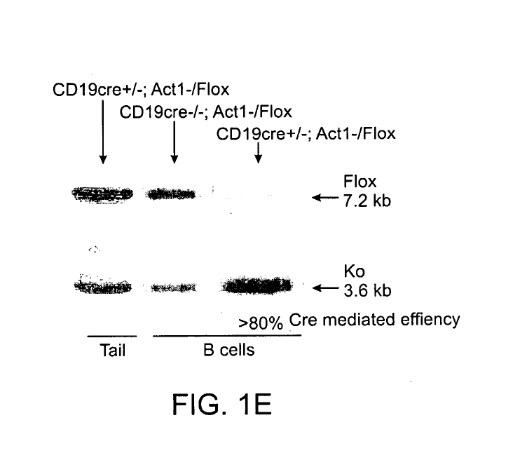

To generate B cell specific Act1 knockout mice, the Act1-deficient mice were bred onto CD19cre mice (CD19cre⁺/⁺) to generate CD19cre^{+/-}Act1^{+/-} mice. These mice were further bred onto Act1 floxed mice (Act1^{flox/flox}) to generate control mice (CD19cre^{+/-}Act1^{+/flox}) and B cell specific knockout mice (CD19cre^{+/-}Act1^{+/flox}). CD19cre mediated deletion efficiency was determined by southern blot with the probe (prb3) shown in FIGS. 1A-1D.

Generation of the CD40-Act1 and BAFF-Act1 Double Knockout Mice

To generate the double knockout mice, Act1 complete knockout mice (Act1^{-/-}) were first bred onto CD40 knockout mice (CD40^{-/-}) (purchased from Jackson Laboratory; Kawabe, T., et al., Immunity 1:167-178 (1994)) or BAFF knockout mice (BAFF^{-/-}) to first generate heterozygous mice (Act1^{+/-}CD40^{+/-} or Act1^{+/-}BAFF^{+/-}), then CD40-null Act1 heterozygous mice (CD40^{-/-}Act1^{+/-}) and BAFF-null Act1 heterozygous mice (BAFF^{-/-}Act1^{+/-}). These mice were then bred among themselves to generate CD40-Act1 and BAFF-Act1 double knockout mice.

Southern and RNA Blots

Genomic DNA was extracted from ES cells or mouse tail tissue, digested with HindIII or KpnI/SpeI (New England Labs), separated by 1% agarose gel electrophoresis and analyzed with either a 5′ external (prb1 for DNA digested with KpnI/SpeI) or a 3′ external probe (prb2 for DNA digested with HindIII). For RNA analysis, total RNA was isolated using Trizol regent (Invitrogen) according to the manufacturer's instruction.

Western Blot Analysis

Splenic B cells were untreated or treated 2 μg/ml anti-mouse CD40 antibody (BD pharmingen), 2 μg/ml anti-IgM (Jackson Immuno-Research), 10 ng/ml TNFα (Peprotech), 0.1 μg/ml mouse BAFF ligand (Apotech Biochemicals, Epalinges, Switzerland) or 0.2 μg/ml mouse April ligand (generously provided by Amgen Inc., California) for different time points. Western analyses were performed with antibodies against phospho-JNK, JNK, phospho-IκBa, IκBa, phospho-ERK, ERK, phospho-P38, P38 (Cell Signaling, Beverly, Mass.) and CD40, Bcl2, Bcl-x_{L}, α-tubulin, Actin (Santa Cruz Biotechnology, CA) and P100/P52 (kindly provided by Dr. Green, La Jolla Institute for Allergy and Immunology, San Diego, Calif.).

B Cell Preparation and B Cell Survival Assay

Total mouse splenic B cells were purified by two-rounds of panning with anti-B220. The resting splenic B cells were then purified from the total B cells through 50%, 60% and 70% Percoll gradient centrifugation.

For the cell survival assay, splenic B cells from naive mice were incubated at concentration of 3×10⁶ cells/ml in RPMI medium plus 10% FCS, untreated or treated with CD40 antibody or BAFF ligand for 4 days. The annexin V-FITC apoptosis detection kit was used for detecting apoptotic cells and propidium iodide used for detecting dead cells in the flow analysis. The percentage of remaining live cells was determined by flow analysis using a FACS Calibur machine (Becton Dickinson).

Flow Cytometry

Single cell suspension from various tissues was stained with conjugated mAbs (including PE-B220, FITC-CD5, FITC-IgD, PE-IgM, FITC-CD21, Biotin-B220, Biotin-CD23, FITC-GL-7, PE-FAS, FITC-I-A, PE-CD11c, FITC-CD4, PE-CD8, FITC-CD43 and FITC-Thy1 (BD PharMingen). Cell associated fluorescence was analyzed using a FACScan instrument and associated Winlist 5 software.

Isotype- and Antigen-Specific ELISAs

Serum IgM, IgG1, IgG2a, IgG2b, IgG3, IgA and IgE were determined by isotype-specific ELISA (Southern Biotechnology Associates, Birmingham, Ala.). The data were analyzed by Softmax Pro2.1. For T-cell dependent antigen specific IgM, IgG1 and IgG2b, antibody titers were determined as described for isotype-specific ELISAs, except that plates were coated with 5 μg/ml capture antigen of either NP₄-BSA or NP₃₀-BSA. Data for the antigen specific antibodies were presented as reading at 405 nm with serum dilution of 1:100. For T cell independent antigen specific IgM, IgG1, IgG2a and IgG2b, plates were coated with 5 μg/ml capture antigen of NP30-BSA. Autoantibodies to histone, dsDNA and single-stranded DNA (ssDNA) were measured by ELISA as previously described (Vyse, T. J., et al., J. Clin. Invest 98:1762-1772 (1996)).

Immunizations

7-week-old wild type or Act1-deficient mice were immunized intraperitoneally with 20 μg of NP₂₈—CGG for TD response, or 20 mg of NP-Ficoll for TI response (Biosource Technologies, Vacaville, Calif.). On days 10 and 14 after immunization for TD or days 8 and 15 for TI, serum was collected from peripheral blood. Circulating antibodies were measured by an isotype-specific, antigen specific ELISA. NP₃₀-BSA was used to capture all antigen-specific Igs, whereas NP₄-BSA was used to capture only high-affinity Igs. Captured antibodies were detected with enzyme-conjugated rabbit anti-IgM, anti-IgG1, anti-IgG2a and anti-IgG2b.

Immunohistochemistry

Spleens were embedded in OCT compound (Tissue-Tek, Sakura, Torrance, Calif.) and slowly frozen over liquid nitrogen and stored at −90° C. Cryostat sections were incubated with biotinylated anti-B220, anti-CD5, anti-Syndecan-1 (BD Pharmingen, San Diego, Calif.), biotinylated-PNA (Vector Labs, Burlingame, Calif.), or biotinylated anti-MOMA1 (Bachem, King of Prussia, Pa.). Sections were developed using the avidin/biotin system (Vecastain ABC kit, Vector Labs, Burlingame, Calif.) per the manufacturer's instructions. Slides were mounted in Crystal Mount (Biomeda, Foster City, Calif.) and analyzed using a Nikon E800 microscope.

Co-Immunoprecipitations

Primary human splenocytes were isolated from discarded human spleen tissue of cadaver transplant donors and cultured in 110% FCS+RPMI. Human B cell line IM9 is cultured in 10% FCS+RPMI. Immunoprecipitations were performed on these cells as previously described (Qian, Y., et al., Proc. Natl. Acad. Sci. USA 99:9386-9391 (2002)). Antibodies used for immunoprecipitations include CD40 (H1120, Santa Cruz Biotechnology), BaffR (Prosci Inc.), TRAF2 (C20, Santa Cruz Biotechnology), TRAF3 (H1122, Santa Cruz Biotechnology), TRAF6 (H274, Santa Cruz Biotechnology) and Act1 (Qian, Y., et al., Proc. Natl. Acad. Sci. USA 99:9386-9391 (2002)).

Results

Lymphoid system defects in Act1-deficient mice

To generate Act1-null mice, the Act1 targeting construct bearing a disrupted exon 2 (encoding residues 1-268) was transfected into ES cells derived from the 129/sv mouse strain, followed by the transfection of Cre to remove the Neo drug marker and the exon 2 of Act1 (FIG. 1A). Targeted ES clones containing the complete Act1-null allele were injected into blastocysts derived from BALB/c mice (Act1-null allele, FIGS. 1A and 1B). Germline-transmitted chimeras were obtained and bred to BALB/c. Heterozygous mice (F1) were obtained and bred to homozygotes (FIG. 1C).

Act1 expression is induced in mouse tissues upon intraperitoneal injection of lipopolysaccride (LPS) (Zhao, Z., et al., J. Immunol. 170:5674-5680 (2003)). Act1 expression is also induced in mouse splenic B cells upon stimulation with BAFF, CD40L and LPS, implicating its important role in B cell function (FIG. 1D). The LPS-induced expression of Act1 in mouse splenic B cells was no longer detectable in Act1-deficient mice (FIG. 1D). The expression patterns are consistent with a negative modulating role for Act1 during B cell activation.

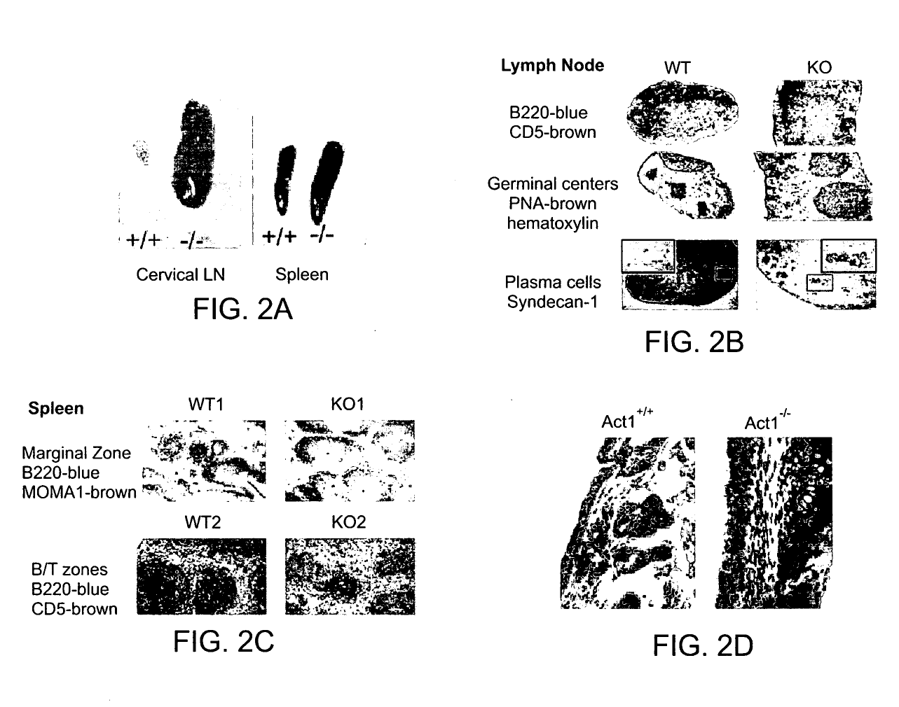

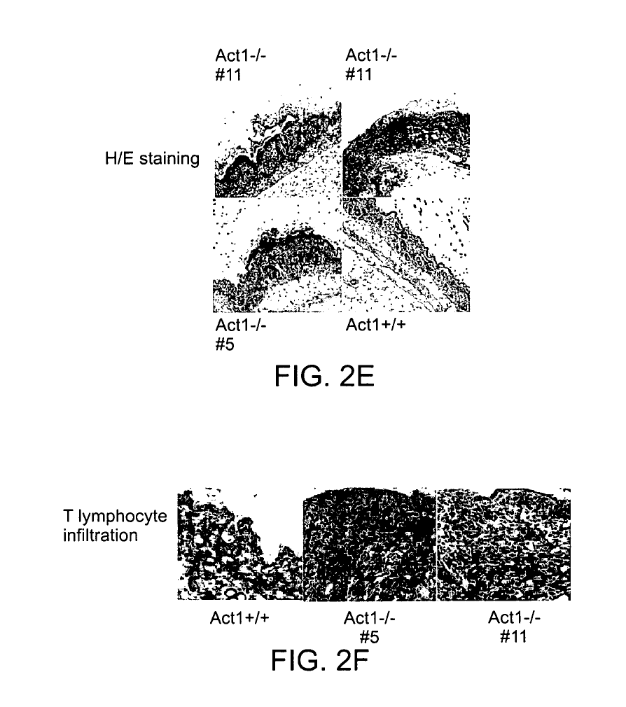

The Act1-deficient mice displayed major lymphoid system abnormalities as early as three-weeks old. The lymph nodes (cervical, axillary and brachial) were massively enlarged (FIG. 2A). Lymphadenopathy is due to lymphoid hyperplasia, increased germinal centers and the accumulation of large numbers of immunoglobulin-producing plasma cells (Syndecan-1 positive cells) in the medulla of the lymph nodes (FIG. 2B). Act1-deficient mice also developed enlarged spleen (FIG. 2A). The microscopic structure of the spleen is largely unaltered, although increased density of B cell zones was observed in some of the Act1-deficient spleens, as compared to wild-type littermate controls (KO2 compared to WT2 in FIG. 2C). Furthermore, the Act1-deficient mice developed inflammation in multiple tissues, including upper respiratory airway and skin (FIGS. 2D-2F).

Hypergammaglobulinemia in Act1-Deficient Mice

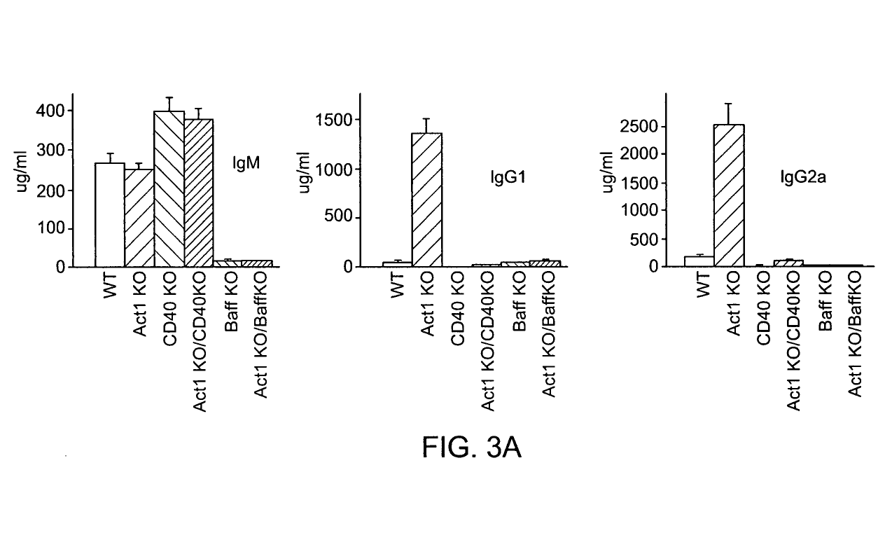

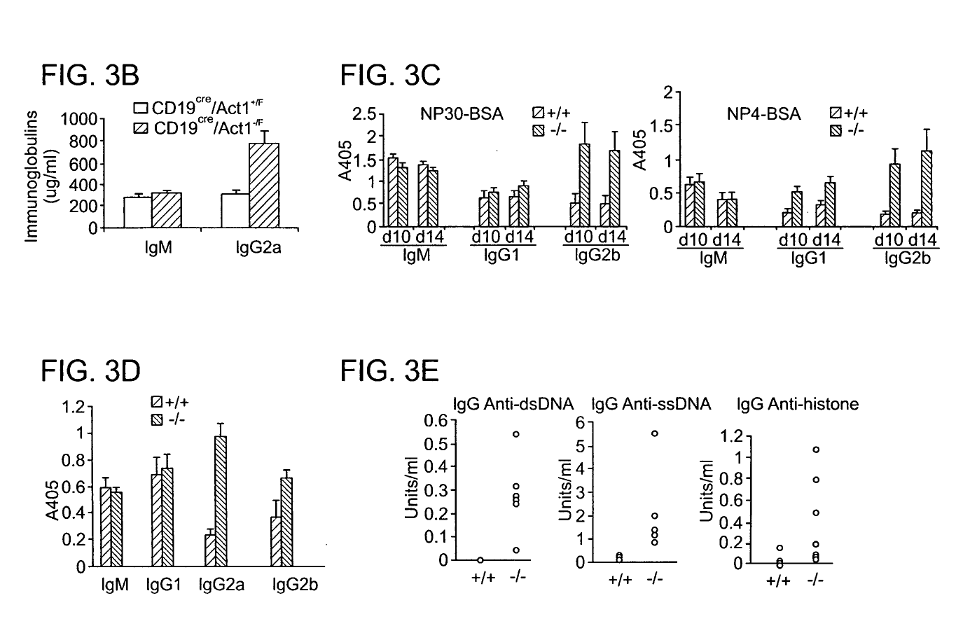

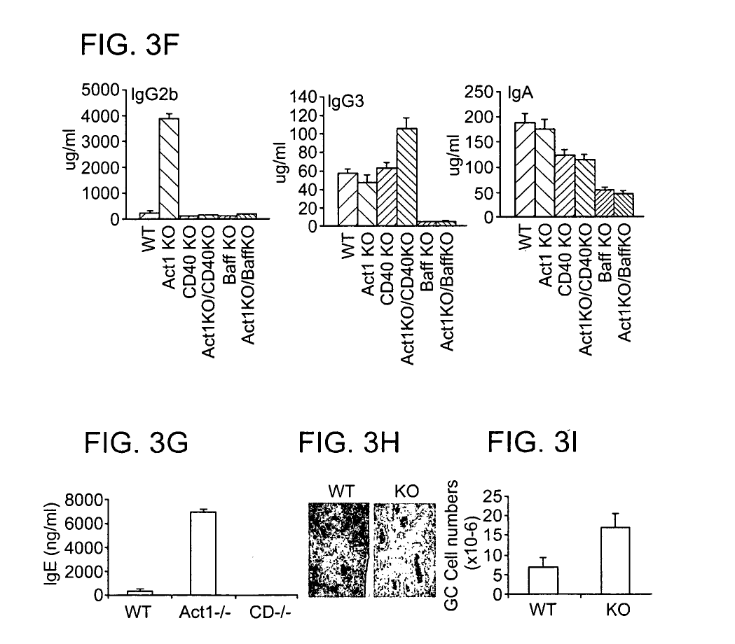

In the CD40-deficient mice, the constitutive levels of immunoglobulins including IgG1, IgG2a, IgG2b and IgE are much lower as compared to wild-type control mice (FIGS. 3F and 3G). In contrast, IgG subclasses and IgE were substantially increased, more than 10-fold, in the Act1-deficient mice, as compared to those in the wild-type control mice. However, the levels of IgM, IgG3 and IgA were not changed in either CD40- or Act1-deficient mice (FIG. 3A and FIG. 3F). The hypergammaglobulinemia in Act1-deficient mice is consistent with the increased numbers of plasma cells in the secondary lymphoid system of the Act1-deficient mice. Interestingly, mice that overexpress BAFF display a very similar phenotype to the Act1-deficient mice, including lymphadenopathy and hypergammaglobulinemia (Gross, J. A., et al., Nature 404:995-999 (2000); Mackay, F., et al., J. Exp. Med. 190:1697-1710 (1999)). Taken together, Act1 likely functions as an important modulator in humoral immune responses, possibly through its involvement in CD40- and/or BAFF-mediated pathways.

To determine whether Act1 indeed exerts its regulatory role in humoral immune responses through the CD40 and BAFF pathways, CD40-Act1 and BAFF-Act1 double knockout mice were generated. Both the CD40-Act1 and BAFF-Act1 knockout mice had much reduced enlargement of lymph nodes and spleen as compared to Act1-deficient mice. The hypergammaglobulinemia observed in Act1-deficient mice was abolished in CD40-Act1 and BAFF-Act1 double knockout mice (FIG. 3A and FIG. 3F). These results strongly indicate that Act1 modulates the humoral immune responses through its interaction with the CD40- and BAFF-mediated pathways.

In Vivo Responses to Antigen Challenge

To examine the role of Act1 in T-cell dependent (TD) antigen-specific responses, the Act1-deficient and littermate control mice were challenged with nitro-phenol-conjugated chicken gamma-globulin (NP₂₈—CGG), and specific antibody responses measured (FIG. 3C). Upon in vivo challenge with NP-CGG, Act1-deficient mice developed much higher titers of both total (NP30-BSA, FIG. 3C) and high-affinity (NP4-BSA, FIG. 3C)NP-specific IgG2b antibodies. Although the total NP-specific IgG1 level was slightly increased (NP30-BSA, FIG. 3C), the Act1-deficient mice developed significantly increased high-affinity IgG1 (NP4-BSA, FIG. 3C). However, NP-specific IgM antibody production was unchanged in Act1-deficient mice. The Act1-deficient mice and littermate control mice were then immunized with the T-cell independent (TI) antigen NP-Ficoll, and specific antibody responses measured. Upon in vivo challenge with NP-Ficoll, Act1-deficient mice developed much higher titers of total NP-specific IgG2a and IgG2b antibodies (FIG. 3D). However, NP-specific IgM and IgG1 production was unchanged in Act1-deficient mice (FIG. 3D). The hyper TD and TI antigen-specific responses observed in Act1-deficient mice suggests that Act1 probably plays a negative regulatory role in both T-cell dependent and independent humoral immune responses, probably through its interaction with the CD40- and/or BAFFR-mediated pathways (Kawabe, T., et al., Immunity 1:167-178 (1994); Xu, J., et al., Immunity 1:423-431 (1994)).

Production of Autoantibodies

Dysregulation of the humoral immune response often leads to autoimmune disease. Both anti-ssDNA and anti-dsDNA IgG antibodies were detected in most of the Act1-deficient mice, but not in the littermate control mice (FIG. 3E). Furthermore, anti-histone IgG antibodies were also detected in the Act1-deficient mice, but not in the littermate control mice. Consistent with the above conclusion that Act1 is involved in BAFF-BAFFR pathway, transgene-mediated overexpression of BAFF causes breakdown of B cell tolerance and leads to production of autoantibodies and a SLE-like condition in mice (Gross, J. A., et al., Nature 404:995-999 (2000); Mackay, F., et al., J. Exp. Med. 190:1697-1710 (1999)).

Increased Peripheral B Cells

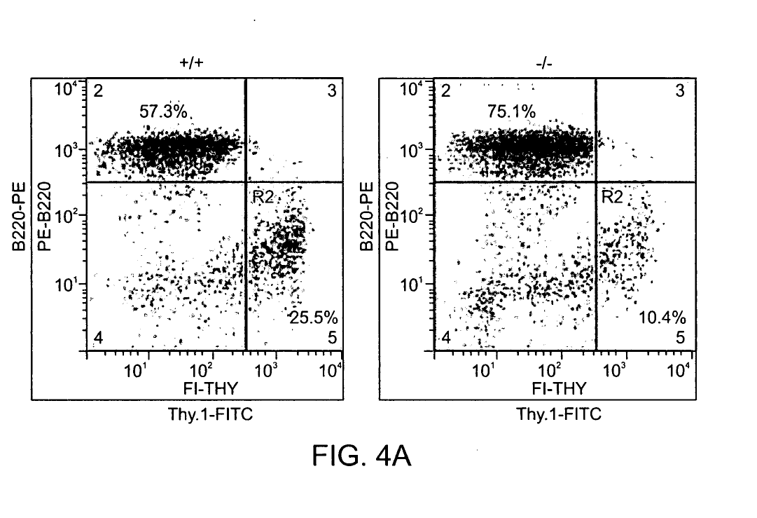

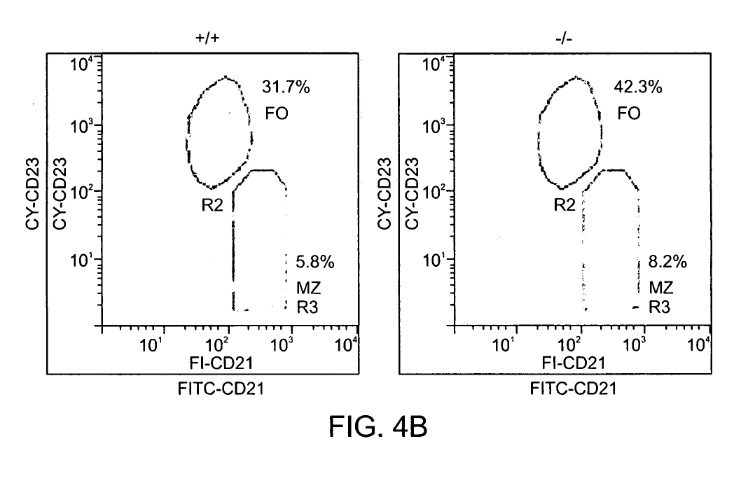

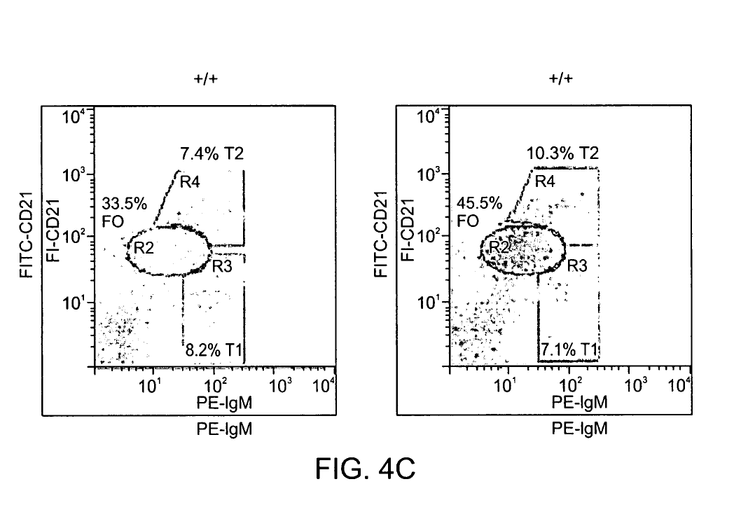

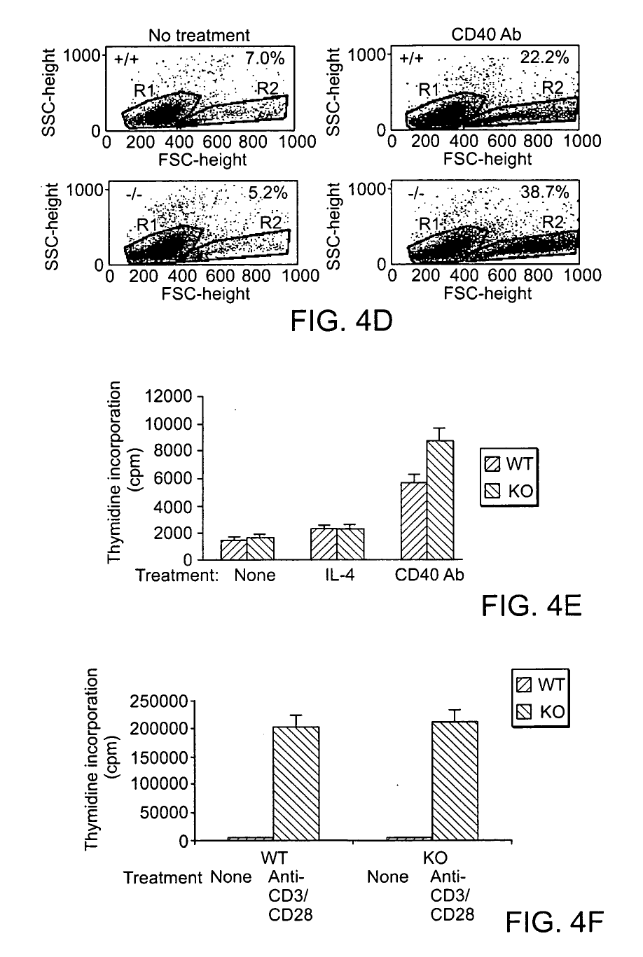

The T and B cell compartments of thymus and spleen of Act1-deficient and littermate control mice by flow cytometry were analyzed. The total T cells (Thy1+) and the proportion of T cells in the subsets defined by CD4 and CD8 cell surface markers were unaltered (Table). However, the total splenic B cell (B220+) population was significantly increased in Act1-deficient spleen, as compared to that of littermate control mice that were gender matched (Table and FIG. 4A). The decreased percentage of T cells in the spleen (Thy.1+, FIG. 4A) was probably due to increased B cells, since the total numbers of T cells per spleen were unaltered (Table). In addition to the increased B cell population, the dendritic cell population (1-A+CD11c+) was also increased in Act1-deficient spleen as compared to the wild-type control (Table).

Analysis of B220+cells in the bone marrow of Act1-deficient mice indicated no changes in pro-B cells (B220⁺CD43⁺IgM⁻), pre-B cells (B220⁺CD43⁻IgM⁺), and immature B lymphocytes (B220⁺IgM⁺IgD⁻) compartments (Table). Thus, lack of Act1 does not affect the generation or differentiation of bone marrow lymphoid progenitor cells that gives rise to fully committed B lymphocytes. As indicated above, B220⁺ B cells in secondary lymphoid organs were significantly increased (Table and FIG. 4A). Analysis of splenic B cells showed elevated mature B cells (B220⁺IgM^{lo}IgD⁺; Table), follicular B cells (B220⁺CD21^{int}CD23^{hi}; FIG. 4B and Table), marginal zone B cells (B220⁺CD21^{hi}CD23^{lo}, FIG. 4B and Table) and T1 (B220⁺IgM^{hi}CD21⁻) and T2 (B220⁺IgM^{hi}CD21+) transitional B cells (FIG. 4C and Table). These data show that the absence of Act1 led to uniform increase in cellular components of the B cell compartment in secondary lymphoid organ. Taken together, these results indicate that Act1 likely plays a critical regulatory role in the survival of peripheral B cells.

TABLE
B Cell, T Cell and Dendritic Cell Populations

| Bone Marrow (×10⁶) | Act1^{+/+} | Act1^{-/-} | Fold |
|---|---|---|---|
| Pro-B cells | 1.6 ± 0.3 | 1.6 ± 0.3 | 1.0 |
| (B220⁺CD43⁺IgM⁻) | | | |
| Pre-B cells | 4.5 ± 0.7 | 4.1 ± 0.6 | 0.9 |
| (B220⁺CD43⁻IgM⁺) | | | |
| Immature B cells | 3.3 ± 0.6 | 2.9 ± 0.6 | 0.9 |
| (B220⁺IgM⁺IgD⁻) | | | |
| Recirculating mature | 0.43 ± 0.1 | 0.77 ± 0.1 | 1.8 |
| B cells | | | |
| (B220⁺IgM^{lo}IgD⁺) | | | |

| Spleen (×10⁶) | Act1^{+/+} | Act1^{-/-} | Fold | CD19cre/Act1^{+/F} | CD19cre/Act1^{-/F} | Fold |
|---|---|---|---|---|---|---|
| Total B cell | 120.9 ± 8.4 | 269.9 ± 5.1 | 2.2 | 105.2 ± 7.4 | 136.3 ± 8.9 | 1.3 |
| Mature B cells | 37.5 ± 1.8 | 102.3 ± 2.0 | 2.7 | 34.9 ± 3.3 | 52.1 ± 4.8 | 1.5 |
| Marginal zone B cells | 5.6 ± 0.4 | 18.4 ± 0.8 | 3.3 | 6.2 ± 0.6 | 12.8 ± 1.3 | 2.1 |
| Follicular B cells | 31.1 ± 1.7 | 77.6 ± 2.1 | 2.5 | 28.9 ± 2.4 | 40.9 ± 4.1 | 1.4 |
| T1 transitional B cells | 8.2 ± 0.3 | 16.0 ± 1.0 | 1.9 | 6.8 ± 0.7 | 9.6 ± 0.9 | 1.4 |
| T2 transitional B cells | 4.1 ± 0.3 | 10.2 ± 0.5 | 2.5 | 3.6 ± 0.4 | 5.5 ± 0.5 | 1.5 |
| Dendritic cells | 6.1 ± 1.0 | 15.8 ± 2.2 | 2.6 | 4.2 ± 0.4 | 5.8 ± 0.7 | 1.4 |
| Total T cells | 37.7 ± 3.0 | 34.2 ± 2.1 | 0.9 | 43.5 ± 4.1 | 43.9 ± 3.7 | 1.0 |
| CD4⁺ cells | 27.1 ± 2.3 | 24.4 ± 3.1 | 0.9 | 31.1 ± 2.8 | 31.6 ± 3.1 | 1.0 |
| CD8⁺ cells | 10.6 ± 1.0 | 9.5 ± 0.5 | 0.9 | 12.4 ± 1.1 | 12.1 ± 1.2 | 1.0 |

Total B cells (B220⁺), mature B cells (B220⁺IgM^{lo}IgD⁺), marginal zone B cells (B220⁺CD21^{hi}CD23^{lo}), follicular B cells (B220⁺CD21^{hi}CD23^{hi}), T1 transitional B cells (B220⁺IgM^{hi}CD21⁻), T2 transitional B cells (B220⁺IgM^{hi}CD21⁺), dendritic cells (I-A⁺CD11c⁺), total T cells (Thy.1⁺). Data are shown as mean±SEM from three pairs of mice each group. Fold stands for the ratio of cell number from Act1 knockout mice to cell numbers from Act1 control mice. BM cell counts represent the number of cells isolated from two hindleg femurs. Mice were age (6-8 weeks) and gender matched.
Specific Deletion of the Act1 Gene in B Cells

To determine the role of Act1 in B cells, the Act1 gene in B cells was specifically deleted. The Act1-deficient (Act1^{-/-}) mice were first bred onto CD19cre transgenic mice [CD19cre^{+/+} (Li, Z. W., et al., J. Immunol. 170:4630-4637 (2003)) to generate CD19cre^{+/-}Act1^{+/-} mice. These mice were further bred onto Act1 floxed mice (Act1^{flox/flox}) to generate the control mice (CD19cre^{+/-}Act1^{+/flox}) and B cell specific knockout mice (CD19cre^{+/-}Act1^{-/flox}). The deletion efficiency mediated by CD19 promoter driven Cre was about 80%, determined by southern blot with the prb3 (FIGS. 1A-1E). It was found that the B cell specific Act1 knockout mice (CD19cre^{+/-}Act1^{-/flox}) had very similar phenotype as the Act1-deficient mice, although with less severity. The CD19cre^{+/-}Act1^{-/flox} mice developed enlarged lymph nodes and spleen. Hypergammaglobulinemia was also detected in these mice (FIG. 3B). While the T cell population was not altered, the total B cell population and all the subsets of B cells were increased in the CD19cre^{+/-}Act1^{-/flox} (Table). These results strongly indicate that Act1 plays an important role in B cells, which is consistent with the phenotype observed in the Act1-deficient mice. Interestingly, the dendritic cell population was also increased in these B cell specific Act1 knockout mice, indicating that the defect in B cells may have an indirect impact on the dendritic cells.

Increased CD40- and BAFFR-Mediated B Cell Survival

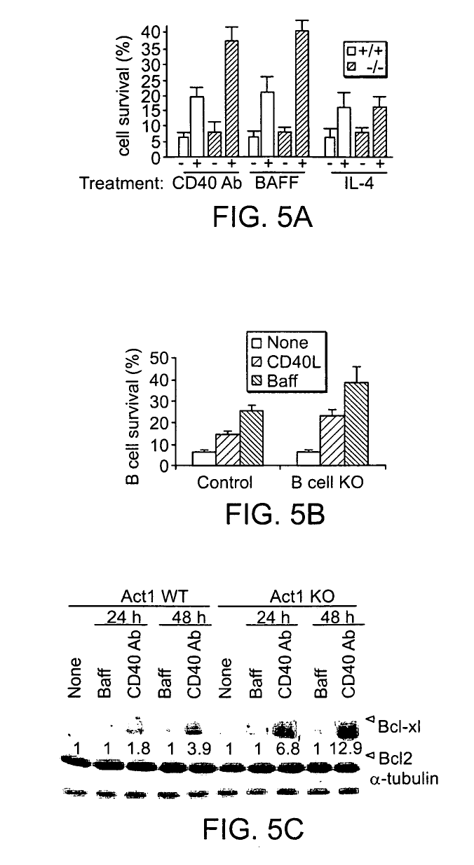

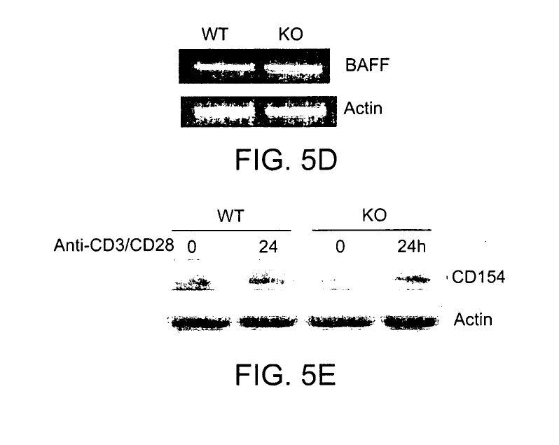

While CD40- and CD40L-deficient mice showed loss of T-cell dependent B cell survival, proliferation and activation, recent studies have also demonstrated the importance of BAFF-BAFFR interaction in B cell survival (Mackay, F., et al., Annu. Rev. Immunol. 21:231-264 (2003); Mackay, F. and Browning, J. L., Nat. Rev. Immunol. 2:465-475 (2002)). The fact that Act1-deficient mice and B cell specific Act1 knockout mice showed increased peripheral B cells implicates a role of Act1 in CD40- and BAFFR-mediated B cell survival. To test this hypothesis, resting mature B cells were purified from spleen and cultured with or without anti-CD40 antibody or BAFF for 4 days. These cultured cells were examined for cell survival by flow cytometry using forward light scatter (FSC)/side light scatter (SSC) plots (Rolink, A. G., et al., Eur. J. Immunol. 32:2004-2010 (2002)) (FIG. 4D). Treatment of wild-type splenocytes with CD40 antibody for 4 days increased the percentage of live cells from 7.0 to 22.2, whereas the same stimulation increased the viability of Act1-deficient splenocytes, from 5.2% to 38.7% (FIG. 5A). BAFF treatment also led to greater increase in cell survival in the Act1-deficient splenocytes, as compared to the cells from littermate control mice (FIG. 5A). However, IL-4- and IgM-mediated B cell survival was unaltered in Act1-deficient mice (FIG. 5A). It is important to point out here that CD40- and BAFF-mediated cell survival was also enhanced in splenic B cells isolated from B cell specific Act1 knockout mice (FIG. 5B), indicating an intrinsic defect in Act1-deficient B cells. The role of Act1 in CD40-mediated B cell proliferation was then examined. CD40-mediated proliferation was increased 40% in Act1-deficient B cells as compared to that in wild-type cells (FIG. 4E), which likely contributes to the enhanced CD40-mediated B cell survival in Act1-deficient mice described above (FIG. 5A).

The prosurvival proteins Bcl-x1 and Bcl2 have been implicated in CD40- and BAFFR-mediated B cell survival (Mackay, F., et al., Annu. Rev. Immunol. 21:231-264 (2003)). Western blot analysis showed that Act1-deficient B cells had much induced levels of Bcl-x1 in response to stimulation with CD40 antibody (FIG. 5C), strongly indicating that Bcl-x1 is probably one of the important effectors responsible for Act 1-regulated CD40-mediated B cell survival.

Increased CD40- and BAFFR-Mediated Signaling

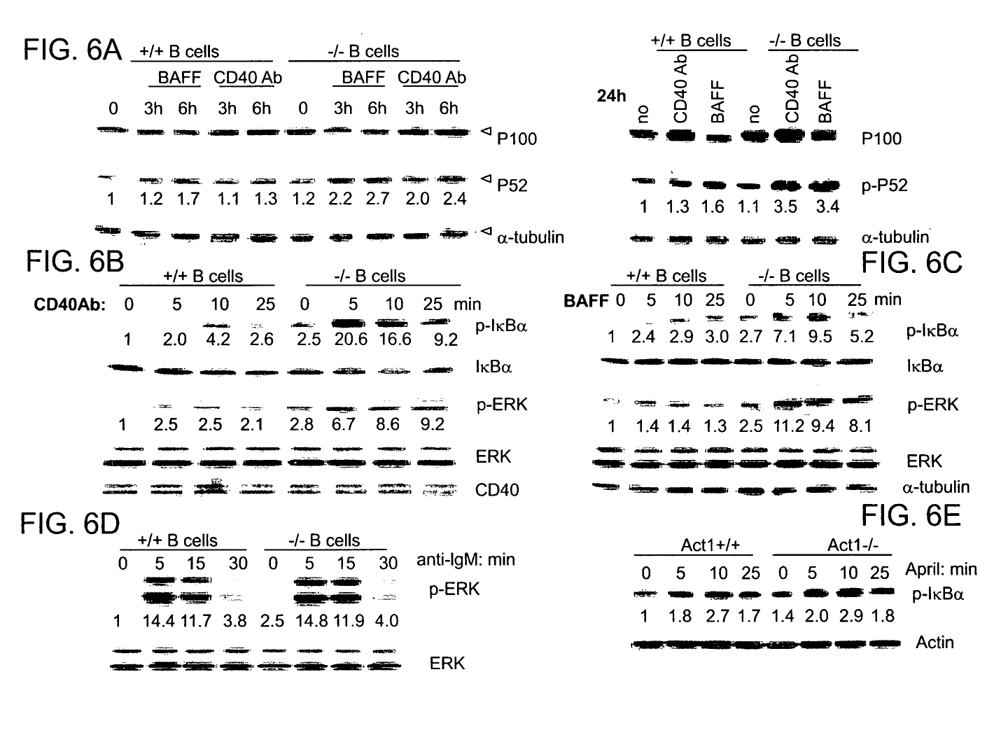

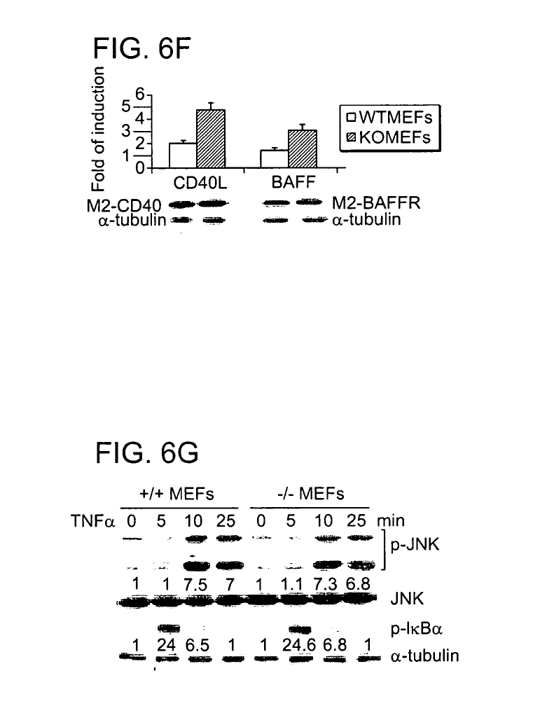

Whether Act1 had any direct effects on the immediate signaling events mediated by CD40 and BAFF was also determined. Act1-deficient B cells showed stronger IκB phosphorylation and enhanced p100 processing to p52 in response to CD40 antibody and BAFF stimulation, implicating a negative regulatory role of Act1 in both canonical and noncanonical NFκB activation pathway (FIGS. 6A-6C). Interestingly, the CD40-induced IκB phosphorylation was much stronger than that induced by BAFF treatment in Act1-deficient B cells. As activation of NFκB (through the canonical pathway) induces the expression of p100, Act1-deficient B cells showed higher levels of NFκB2/p100 precursor in response to CD40 antibody stimulation (FIG. 6A). Furthermore, Act1-deficient B cells showed enhanced activation of MAP kinases, including JNK, ERK and p38 in response to CD40 antibody and BAFF (FIGS. 6B, 6C). It is important to point out here that the expression of CD40-CD40L and BAFF-BAFFR was not altered in Act1-deficient mice as compared to wild-type controls (FIG. 6B, FIGS. 5D-5E). Taken together, these results indicate that Act1 negatively regulates the CD40- and BAFFR-mediated pathways, which likely results in its inhibitory effect on B cell survival. As controls, it was shown that the levels of April—(a ligand for TACI, BCMA but not BAFFR) and BCR-mediated signaling are the same between wild-type and Act1-deficient B cells (FIGS. 6D and E).

In addition to B cells, the role of Act1 in CD40- and BAFFR-mediated signaling was also examined in primary mouse embryonic fibroblasts (MEFs) (FIGS. 6F-6G). Act1-deficient MEFs showed increased CD40- and BAFFR-mediated NFκB-dependent E-selectin promoter activity, indicating that Act1 can also negatively regulate the CD40- and BAFFR-mediated signaling in non-B cells.

Interaction of Act1 with Other Signaling Molecules

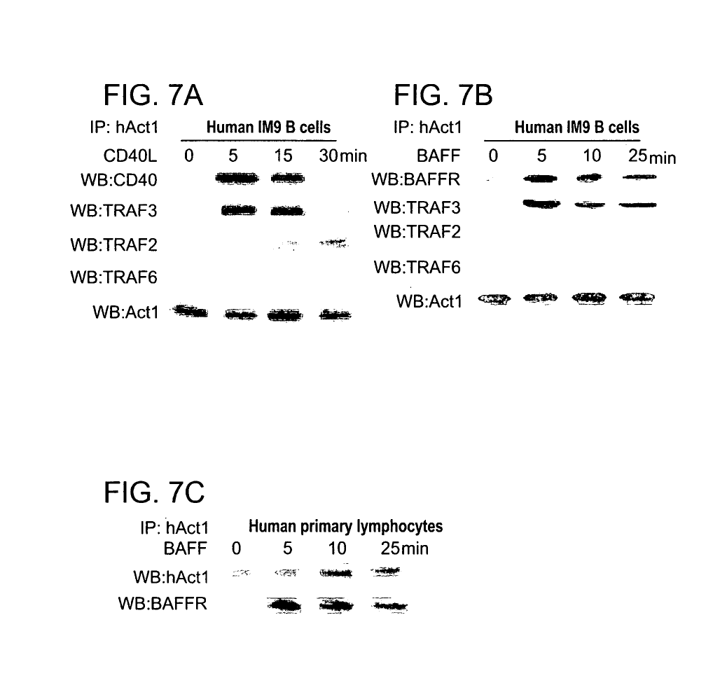

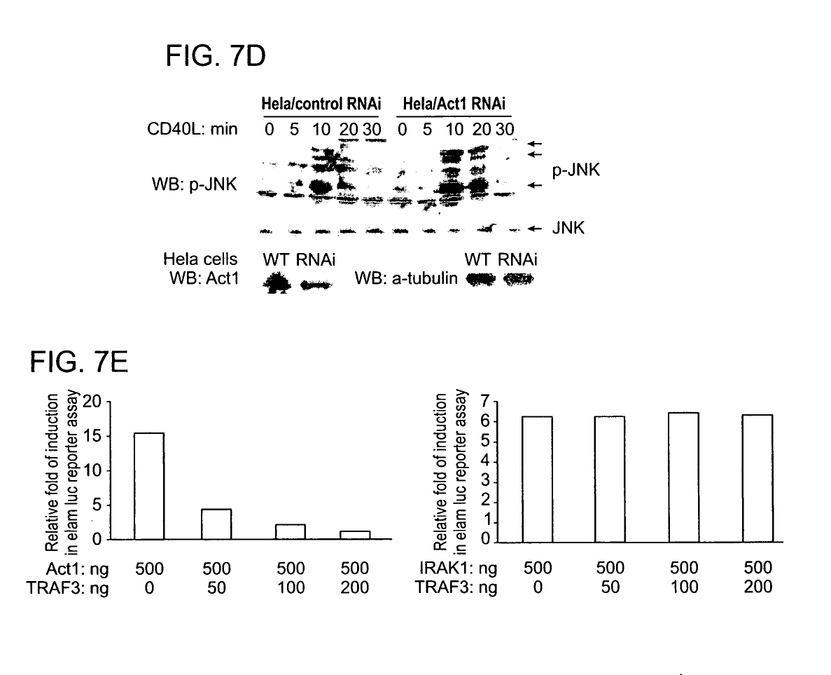

To elucidate the mechanism of action of Act1, its interaction with known components of the CD40 and BAFFR pathway was assessed. Endogenous Act1 was recruited to CD40 and BAFFR upon CD40L and BAFF stimulation in the B cell line IM9 as well as in primary human splenocytes (FIG. 7A-7C). Since TRAF molecules have been shown to function as important adapters for CD40 and BAFFR, the interaction between Act1 and different TRAFs was examined. Previously Act1 was shown to specifically interact with TRAF3, but not with the other TRAFs when they were overexpressed in 293 cells (Qian, Y., et al., Proc. Natl. Acad. Sci. USA 99:9386-9391 (2002)). Under conditions of normal expression endogenous Act1 interacted strongly with TRAF3, weakly with TRAF2, but failed to interact with TRAF6 upon CD40L stimulation as revealed by coimmunoprecipitation (FIG. 7A). On the other hand, endogenous Act1 interacted with TRAF3, but not with TRAF2 or TRAF6 upon stimulation with BAFF (FIG. 7B). Taken together, these preliminary results indicate that Act1 likely regulates the CD40- and BAFFR-mediated signaling through its interaction with the TRAF molecules, particularly TRAF3.

Discussion

As shown herein, CD40- and BAFF-mediated survival is significantly increased in Act1-deficient B cells, implicating a negative or attenuating regulatory role of Act1 in B cell survival. Consistent with this finding, Act1-deficient mice revealed a general increase in peripheral B cells, culminating in lymphadenopathy, splenomegaly, hypergammaglobulinemia, inflammation in multiple tissues, and the formation of autoantibodies. While the B cell specific Act1 knockout mice displayed similar phenotype, the SLE-like pathologies of the Act1-deficient mice were blocked in CD40-Act1 and BAFF-Act1 double knockout mice.

The enhanced CD40- and BAFF-induced p100 processing to p52 in the Act1-deficient B cells is likely to be important for the expanded B cell compartment in the Act1-deficient spleen. Nevertheless, the enhanced canonical NFκB activation pathway and the activation of MAP kinases probably also contribute to the increased survival of Act1-deficient peripheral B cells. The control of cell survival is believed to rely on the regulation of key antiapoptotic and proapoptotic regulators. Previous studies have shown that anti-apoptotic proteins Bcl2 and Bcl-x1 play important roles in B cell survival. Interestingly, it was found herein that stimulation with anti-CD40 resulted in much higher levels of Bcl-x1 in the Act1-deficient B cells than in the wild-type control cells. These elevated levels of Bcl-x1 provides a mechanism for the increased CD40-mediated B cell survival in the Act1-deficient mice. BAFF stimulation did not increase the levels of Bcl-x1 indicating that B cell survival is probably not dependent on this antiapoptotic factor in Act1-deficient mice. While Act1-deficient B cells showed similar levels of p100 processing in response to CD40 antibody and BAFF, the CD40-induced IκB phosphorylation was much stronger than that induced by BAFF treatment. The differential effects of Act1 on CD40- and BAFF-mediated pathways probably result in altered expression of different target genes in Act1-deficient cells.

The fact that Act1 specifically interacts with TRAF3 upon stimulation with CD40L and BAFF indicates that TRAF3 may play an important role in Act1-mediated negative regulation. Interestingly, TRAF3 has been shown to play a negative regulatory role in both CD40 and BAFFR signaling (Xu, L. G. and Shu, H. B., J. Immunol. 169:6883-6889 (2002); Hostager, B. S., et al., J. Biol. Chem. 278: 45382-45390 (2003); Xie, P., et al., J. Exp. Med. 199:661-671 (2004); Liao, G., et al., J. Biol. Chem. 279: 26243-26250 (2004)). TRAF3−/− B cells displayed enhanced CD40-mediated JNK activation. Furthermore, in TRAF3−/− B cells, the recruitment of TRAF2 to CD40 was increased upon CD40 engagement, indicating that TRAF3 may exert its inhibitory effects on CD40 signaling by competing with TRAF2 for association with CD40. Moreover, TRAF3 was shown to interact with NIK, targeting NIK for degradation by the proteasome and inhibiting NIK-mediated p100 processing. It is likely that Act1 exerts its inhibitory role in CD40 and BAFFR signaling through its interaction with TRAF3, by either competing with the positive regulators (such as TRAF2) for interactions with receptors and kinases or targeting the positive regulators for degradation through the proteasome pathway. Interestingly, the expression of both Act1 and TRAF3 are increased in mouse splenic B cells upon stimulation with BAFF, CD40L and LPS (Grammer, A. C. and Lipsky, P. E., Adv. Immunol. 76, 61-178 (2000)), which indicates that these two molecules work together as a feedback control to dampen signaling for B cell survival and activation, regulating homeostasis of B cell functions.

While it has now been clearly shown that Act1 plays a negative role in CD40 and BAFF-mediated pathway, our previous studies showed that overexpression of Act1 can lead to constitutive NFκB activation. One possibility is that Act1 may play differential functions in different cell types. However, it was found herein that CD40- and BAFF-mediated signaling is also elevated in Act1-deficient MEFs, indicating that Act1 is able to function as a negative regulator in cell types other than B cells. Furthermore, siRNA knocking down of Act1 in Hela cells led to enhanced CD40-mediated JNK activation, indicating that Act1 also functions as a negative regulator in cultured epithelial cells (FIGS. 7D-7E). Recent results suggest that the Act1-mediated constitutive NFκB activation is likely due to nonspecific protein-protein interaction carried out by overexpression of Act1. Interestingly, when Act1 and TRAF3 were co-transfected into 293 or Hela cells, TRAF3 specifically and completely inhibited Act1 mediated NF B activation, whereas TRAF3 had no effect on IRAK mediated NFκB activation (FIGS. 7D-7E). This result indicates that when Act1 is in complex with its natural interaction partner (TRAF3), Act1 does not lead to NFκB activation. Upon Act1 overexpression, Act1 probably interacts nonspecifically with other signaling molecule, resulting in constitutive NFκB activation.

In addition to its role in CD40 and BAFFR signaling in B cells, Act1 likely also plays a role in other cell types in signaling events mediated by other members of the TNFR superfamily, especially the subset of TRAF3-utilizing TNFRs. The fact that the dendritic cell population is increased in Act1-deficient mice indicates a potential role of Act1 in dendritic cell maturation and survival. Although the T cell population and proliferation is not affected in Act1-deficient mice (FIG. 4F), possible functions of Act1 in T cells cannot be completely excluded.

### Example 2

### Act1 Mediated Hyper-T Cell Dependant Response is CD40 Dependent

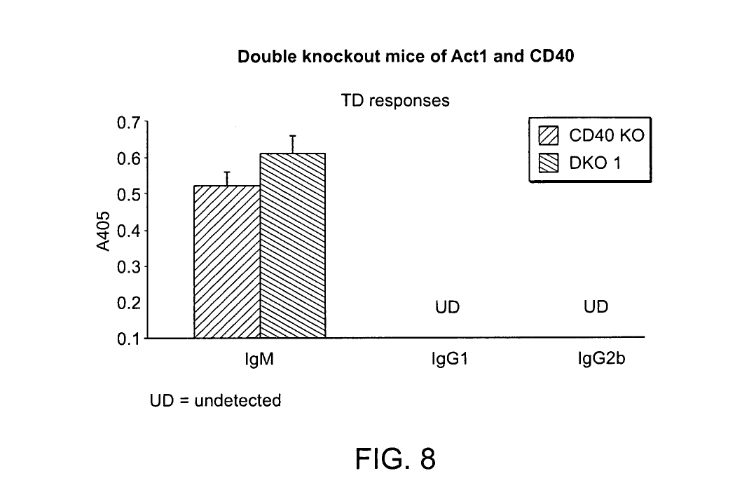

CD40 single knockout mice (CD40KO, N=4) or CD40 and Act1 double knockout mice (DKO1, N=4) were immunized intraperitoneally with 20 ug of NP28-CGG for T cell dependent response. Serum was collected from peripheral blood 12 days after immunization. The NP-specific antibody production was measured by ELISA with the capture antigen NP30-BSA. The data are presented as OD reading at 405 nm. The dilution of sera for the ELISA is 1:1000 for IgM, and 1:100 for IgG1 and IgG2b. As shown in Example 1, the Act-1 deficient mice developed much higher titers of NP-specific IgG1 and IgG2b antibodies as compared to wild type littermate control mice upon in vivo challenge with NP-CGG (Qian, Y., et al., Immunity, 21:575-587 (2004)). As shown in FIG. 8, in response to this T-cell dependent antigen, the hyper NP specific IgG1 and IgG2b antibody response is completely blocked when the Act1-deficient mice were bred onto CD40-deficient mice (resulting in DKO1), whereas the CD40/Act1 double knockout mice (DKO1) produced similar IgM levels as the CD40 single knockout mice (CD40KO). These results indicate that CD40 is required for Act1 mediated hyperimmunoglobulin isotype switching.

### Example 3

### Act1 Mediated Hyper-T Cell Independent Response is CD40 Independent

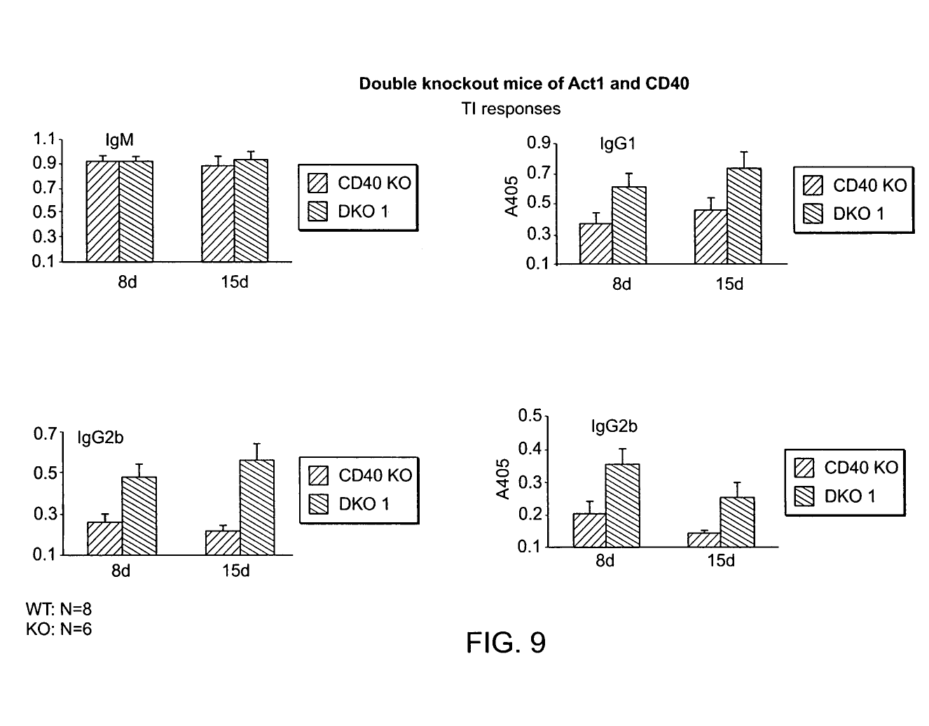

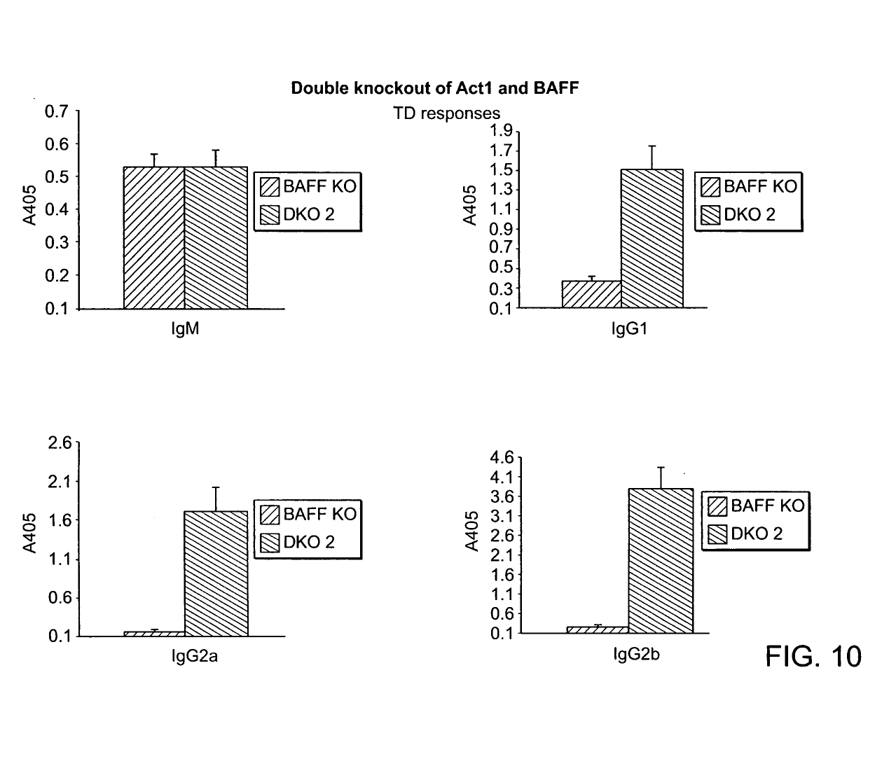

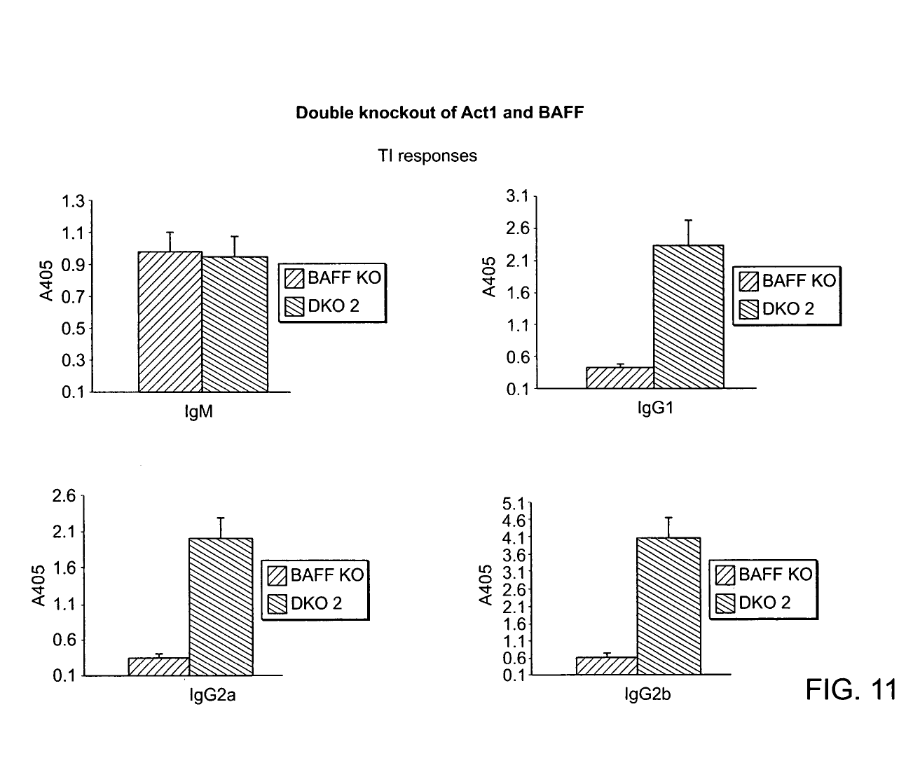

CD40 single knockout (CD40KO, N=8) or CD40 and Act1 double knockout mice (DKO1, N=8) were immunized intraperitoneally with 20 ug of NP-Ficoll for T cell independent response. Serum was collected from peripheral blood for 8 days or 15 days after immunization. The NP-specific antibody production was measured by ELISA with the capture antigen NP30-BSA. The data presented as OD reading at 405 nm. The dilution of sera for the ELISA is 1:1000 for IgM, and 1:100 for IgG1, IgG2a and IgG2b. It was shown in Example 1 that the Act1-deficient mice developed much higher titers of NP-specific IgG1, IgG2a and IgG2b antibodies as compared to wild-type littermate control mice upon in vivo challenge with NP-Ficoll (Qian, Y., et al., Immunity, 21:575-587 (2004)). As shown in FIG. 9, in response to this T cell independent antigen, the CD40/Act1 double knockout mice (DKO1) still developed hyper NP specific IgG1, IgG2a and IgG2b antibodies as compared to CD40-deficient mice (CD40KO), whereas the IgM levels are similar in CD40/Act1 DKO (DKO1) and CD40KO mice. These results indicate that Act1-mediated hyper-T cell independent antibody response is CD40 independent.

### Example 4

### Act1 Mediated Hyper-T Cell Dependent Response is BAFF Independent

BAFF single knockout mice (BAFFKO, N=8) or BAFF and Act1 double knockout mice (DKO2, N=8) were immunized intraperitoneally with 20 ug of NP28-CGG for T cell dependent response. Serum was collected from peripheral blood 12 days after immunization. The NP-specific antibody production was measured by ELISA with the capture antigen NP30-BSA. The data are presented as OD reading at 405 nm. The dilution of sera for the ELISA is 1:1000 for IgM, and 1:100 for IgG1, IgG2a and IgG2b from BAFF single knockout mice while 1:500 for IgG1, IgG2a and IgG2b for the double knockout mice.

It was previously shown that BAFF is required for T-cell dependent antibody response (Schiemann, B., et al., Science, 293:2111-2114 (2001)). As expected, the BAFF single knockout mice (BAFF KO) produced low levels of NP-specific IgM, IgG1, IgG2a and IgG2b antibodies upon in vivo challenge with NP-CGG. Surprisingly, the BAFF/Act1 double knockout mice (DKO2) produced much higher levels of NP-specific IgG1, IgG2a and IgG2b as compared to those in the BAFF single knockout mice, indicating that Act1-mediated T cell dependent antibody response is BAFF independent. Both the BAFF single knockouts and the double knockouts of BAFF and Act1 produced similar IgM levels in response to T-cell dependent antigen.

### Example 5

### Act1 Mediated Hyper-T Cell Independent Response is BAFF Independent

BAFF single knockout mice (BAFFKO, N=8) or BAFF and Act1 double knockout mice (DKO2, N=8) were immunized intraperitoneally with 50 ug of NP-Ficoll for T cell independent response. Serum was collected from peripheral blood 12 days after immunization. The NP-specific antibody production was measured by ELISA with the capture antigen NP30-BSA. The data are presented as OD reading at 405 nm. The dilution of sera for the ELISA is 1:1000 for IgM, and 1:100 for IgG1, IgG2a and IgG2b from BAFF single knockout mice while 1:500 for IgG1, IgG2a and IgG2b for the double knockout mice.

Both the BAFF single knockouts (BAFFKO) and the double knockouts of BAFF and Act1 (DKO2) produced similar IgM levels in response to T-cell independent antigen. Due to the requirement of BAFF for T cell independent response, BAFF single knockout mice produced low levels of NP-specific antibodies including IgM, IgG1, IgG2a and IgG2b. Interestingly, however, the double knockout mice of BAFF and Act1 (DKO2) produced much higher levels of NP-specific IgG1, IgG2a and IgG2b, indicating that Act1-mediated T cell independent response is also BAFF independent.

### Example 6

### Act1 Deficient Mice Developed Sjögren's Syndrome-Like Autoantibodies

Mouse SSA-(Ro) or SSB0 (La) coated plates were analyzed with 1:100 dilution of mouse serum from different age groups (3.5 months or 6-11 months) of Act1 wild type (n=8) or Act1 deficient mice (n=13) in mixed Balb/c/129 background. Both positive and negative controls were provided by the supplier. The optical density (OD) at 450 nm for each serum is derived after subtraction of blank.

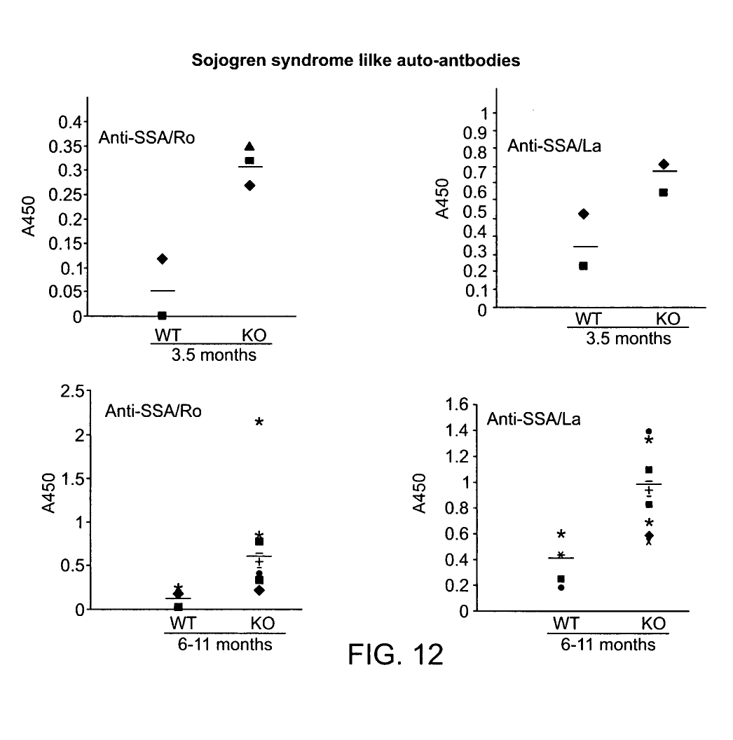

As shown in FIG. 12, the Act1-deficient mice (KO) developed significantly higher levels of anti-SSA/Ro and anti-SSB/La auto-antibodies as compared to the wild type littermate control as early as 3.5 months of age.

### Example 7

### Survival Curve of Act1 Deficient Mice

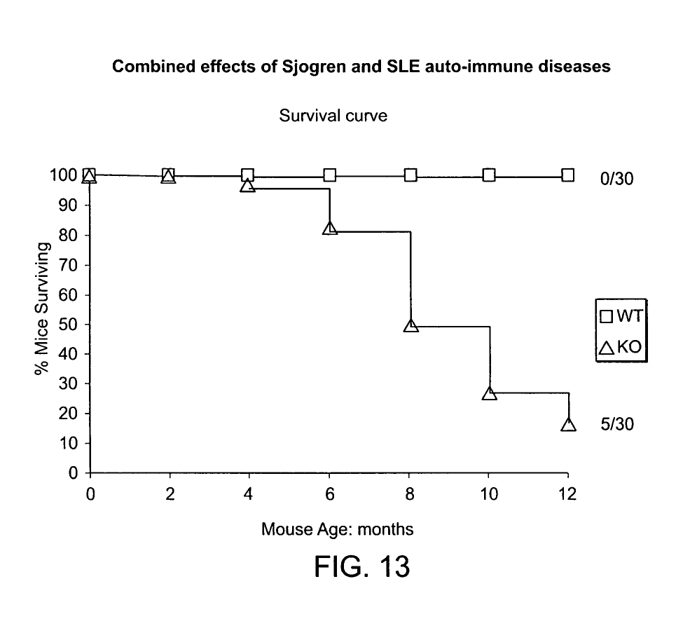

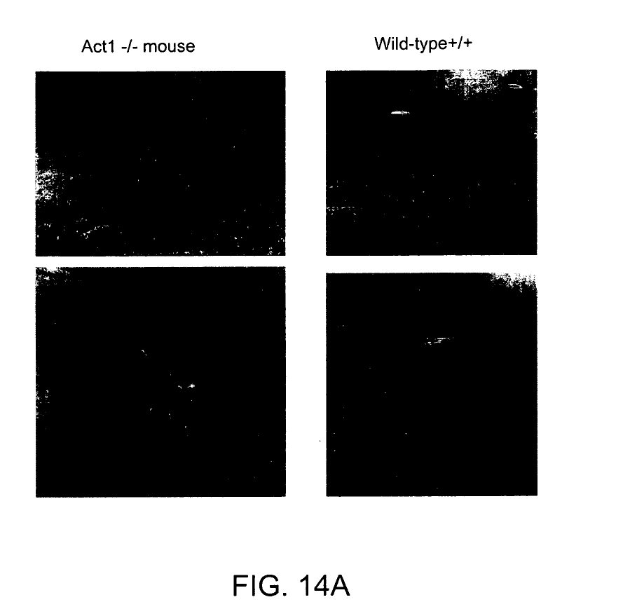

The Act1 deficient mice (KO) exhibit morbidity and mortality, probably due to their Sjögren's and “SLE”-like autoimmune diseases. As shown in FIG. 13, 80% of Act1 deficient mice died by the end of one year of age (5 of 30 mice survived), whereas all of the wild type littermate control mice were alive after one year (all 30 mice survived).

### Example 8

### The Act1-Deficient Mice Tend to Have Difficulties in Maintaining Fully Opened Eyelids Beginning Around 3 Months of Age

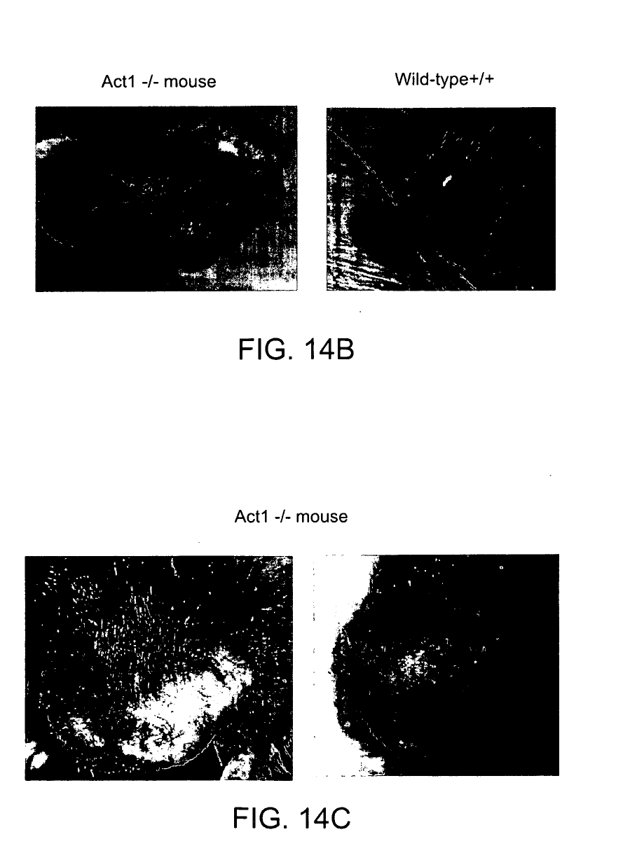

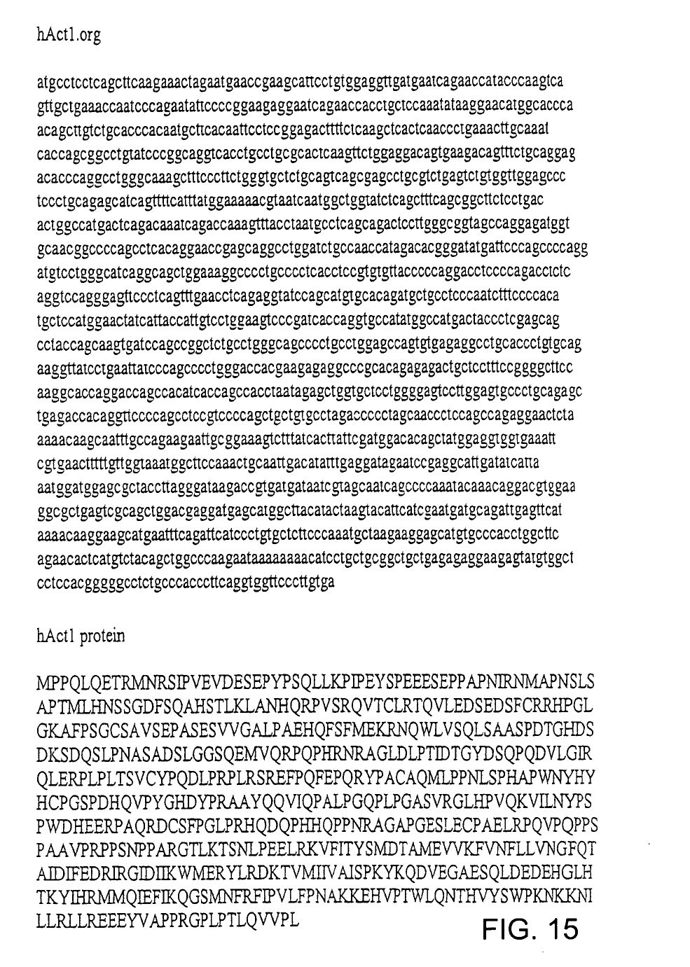

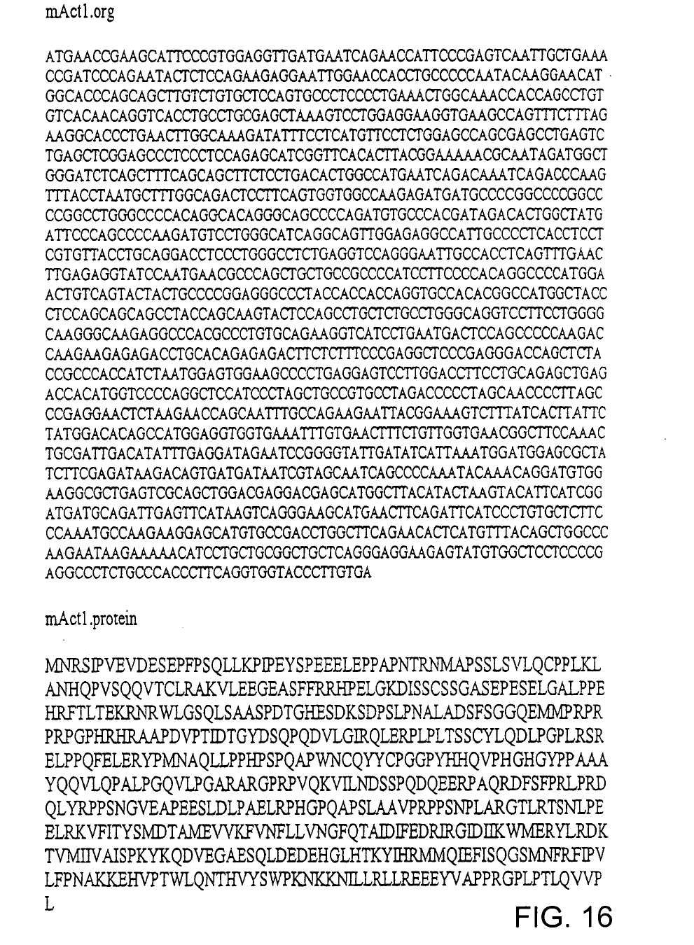

As shown in FIGS. 14A-14C, around 6-8 months, the Act1-deficient mice developed skin lesions around the eyes and mouth, signs of Sjögren's syndrome and “SLE”-like syndrome.

### Example 9

### Act1-Deficient Mice Develop Spontaneous Cancers

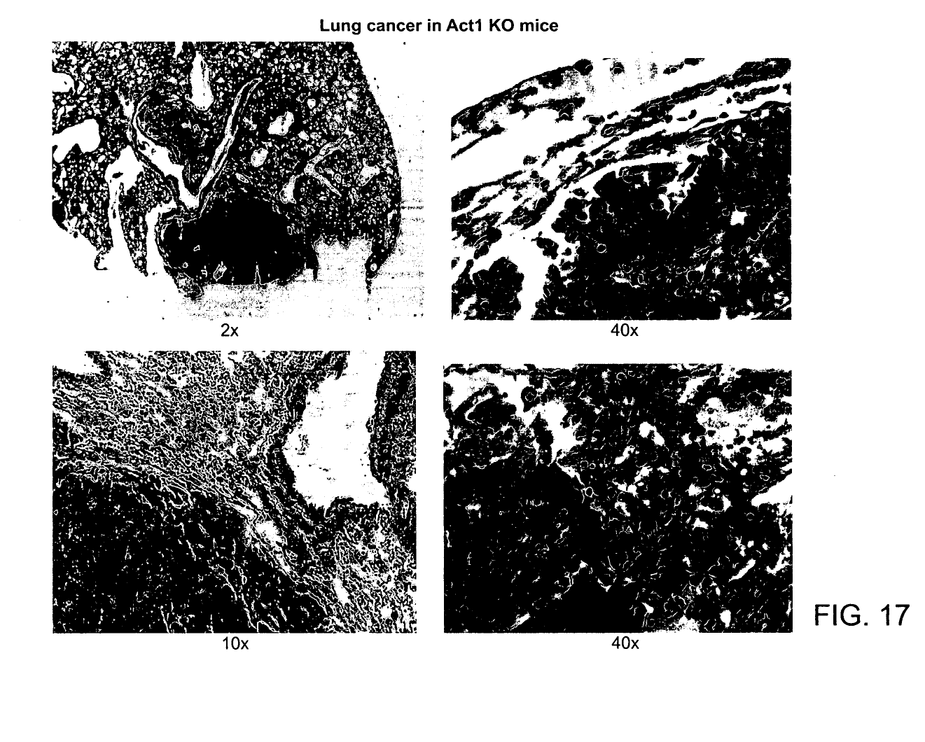

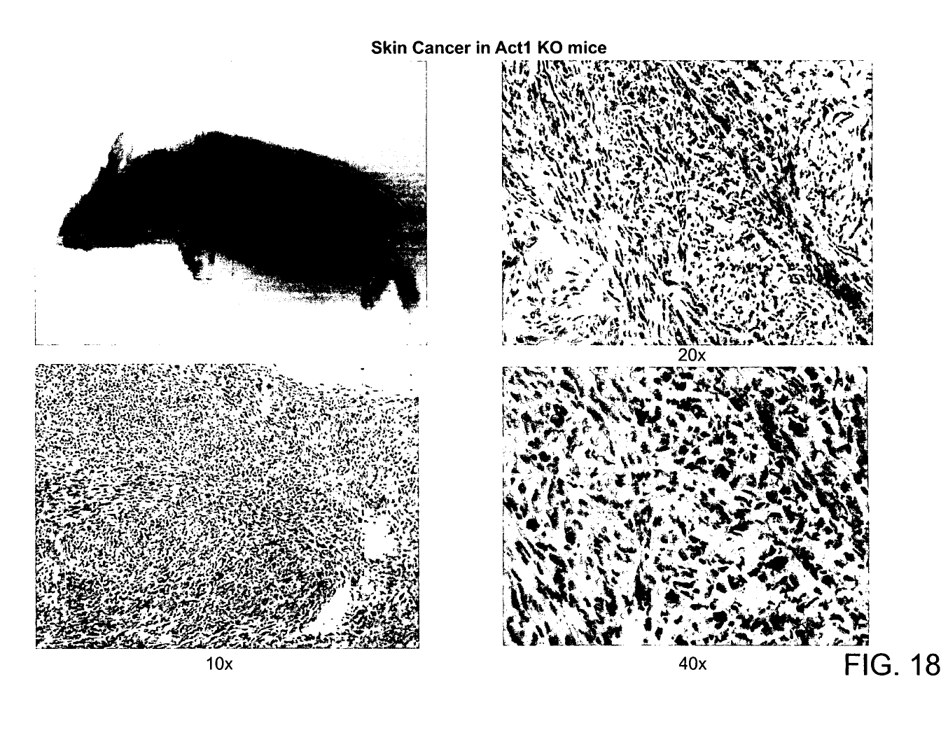

Aged (10-12 months) Act1 deficient mice backcrossed into C57BL/6 background for 5 generations spontaneously developed lung adenoma (FIG. 17) and skin fibroepithelioma (FIG. 18).

### Example 10

### IL-17 Signaling Pathway in Act1-Deficient Mice

Methods

Cell Culture and Biological Reagents

Primary Act1 wild-type and Act1 deficient MEFs were isolated from embryos at day 14.5 and maintained in Dulbecco's modified Eagle's medium, supplemented with 10% fetal calf serum, penicillin G (100 ug/ml), and streptomycin (100 ug/ml). The 293 cells were maintained in the same medium as MEFs. TGFβ activating kinase (TAK1)-deficent MEFS were from Dr. Shiuo Akira, Osaka University. Recombinant IL-17, TNF alpha, IL-1 were from purchased from R & D systems. Antibodies to Traf3, Traf6, human Act1, and actin were from Santa Cruz. Anti-TAK1 was a kind gift of Eli Lilly and company. Anti-flag (M2) was from Sigma. Antibodies against phospho-JNK, phospho-ERK and IκB were from Cell Signaling.

Transfection and Coimmunoprecipation

Cells were transfected by FuGENE (Roche Biomedical Laboratories) according to the manufactuer's instructions. Cell extracts were incubated with 1 μg Ab and 20 μl protein A or M2 beads (Sigma). After overnight incubation, beads were washed four times with lysis buffer, separated by SDS-PAGE and analyzed by immunoblot.

Constructs

The pCMV-Act1 (hemagglutinin (HA)-tagged) has been described (Qian et al., Proc. Natl. Acad. Sci. USA, 99:9386-9391 (2002)). The human IL-17R (flag-tagged) full-length cDNA and the IL-17R deletion mutant (337-534 aa) were generated by PCR and cloned into pcDNA3.1 (Invitrogen).

Animals

Act1-deficient mice were prepared as been described herein (Qian et al., Immunit, 21:575-587 (2004)). To generate epithelia-specific Act1-deficient mice in which the Act1 gene is only deleted from the epithelial cells of the mice (wherein the disruption of the Act1 gene is specific to the epithelial cells), the Act1-deficient mice were bred onto K18cre mice (K18Cre+/+) to generate K18cre+/−Act1+/−mice. These mice were then further bred onto Act1 floxed mice (Act1flox/flox) to generate control mice (K18cre+/−Act1+/flox) and epithelia-specific knockout mice (K18cre+/−Act1−/flox). The Cleveland Clinic Foundation Animal Research Committee approved all of the animal protocols used in this study.

Injections

Mice were injected intraperitoneally with 1 μg IL-17 or 200 ng TNF or combination of both. For intranasal injection experiment, 6 μg IL-17 or PBS were administrated into each mice.

Experimental Autoimmune Encephalomyelitis (EAE) Induced by Active Immunizations

Female C57BL/6 (B6) wild-type (+/−) and Act1 deficient (−/−) mice (10 week old) were injected subcutaneously with an emulsion consisting of 150 μg of a myelin oligodendrocyte glycoprotein 33-35 peptide (MOG 33-35) MEVGWYRSPFSRVVHLYRNGK (SEQ ID NO: 4) in complete Freund's adjuvant (CFA) containing 400 μg mycobacterium tuberculosis H37RA (Difco). Upon immunization (day 0) and two days later, 200 ng of pertussis toxin (List Biological Laboratories) was injected intraperitoneally (i.p.) All mice were weighted and scored daily for neurologic signs according to the following scale: 0, no disease; 1, descreased tail tone or slight clumsy gait; 2, tail atony and/or moderately clumsy gait and/or poor righting ability; 3, limb weakness; 4, limb paralysis, 5, moribund state or death.

Results

IL-17 Mediated Production of Proinflammatory Cytokines are Abolished in Act1 Deficient Mouse Embryo Fibroblasts (MEFs)

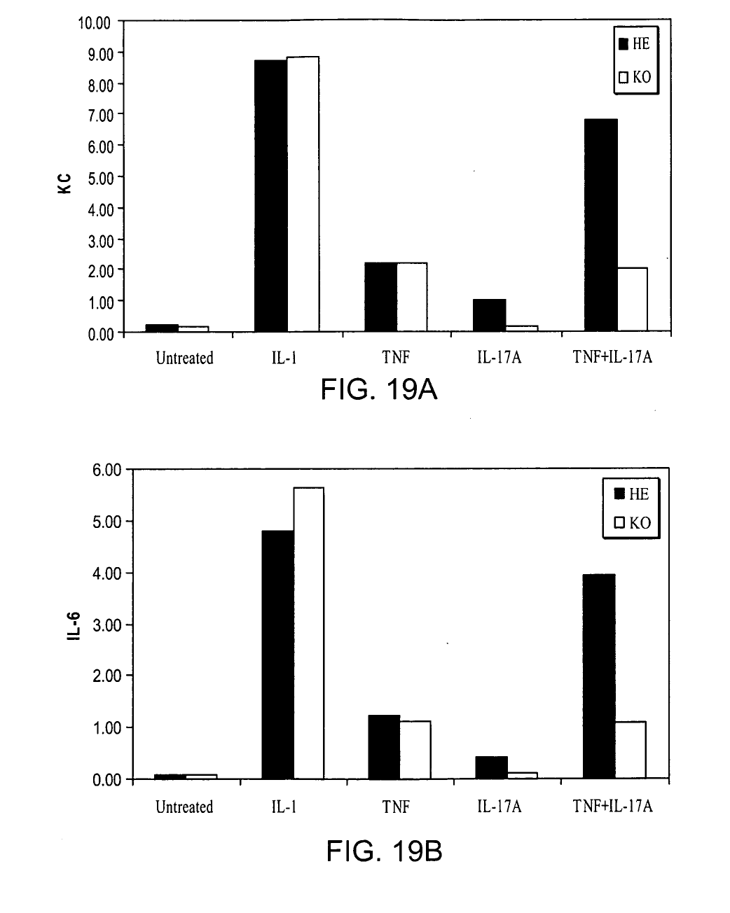

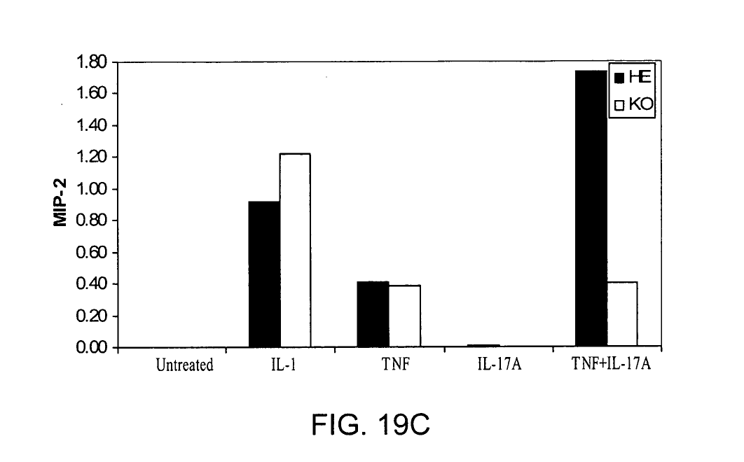

It has been well documented that IL-17 can induce proinflammatory cytokine gene expression alone and in synergy with TNF. To determine if Act1 is a key component in IL-17 signaling pathway, production of several proinflammatory cytokines (KC, IL-6, MIP-2) after treatment of IL-1, TNF, IL-17 or combination of IL-17 and TNF were analyzed in both Act1 deficient and wild-type control MEFs (FIGS. 19A, 19B, 19C). Interestingly, IL-1 and TNF mediated cytokine production showed no difference between Act1 deficient and wild-type control cells, indicating that Act1 did not play a role in the signaling pathways of IL-1 and TNF. In contrast, the cytokine production induced by IL-17 alone or in synergy with TNF was completely abolished in Act1 deficient MEFs, indicating that Act1 is a key component in IL-17 signaling pathway.

IL-17 Mediated Proinflammatory Cytokine Production and Inflammatory Responses are Impaired in Act1-Deficient Mice

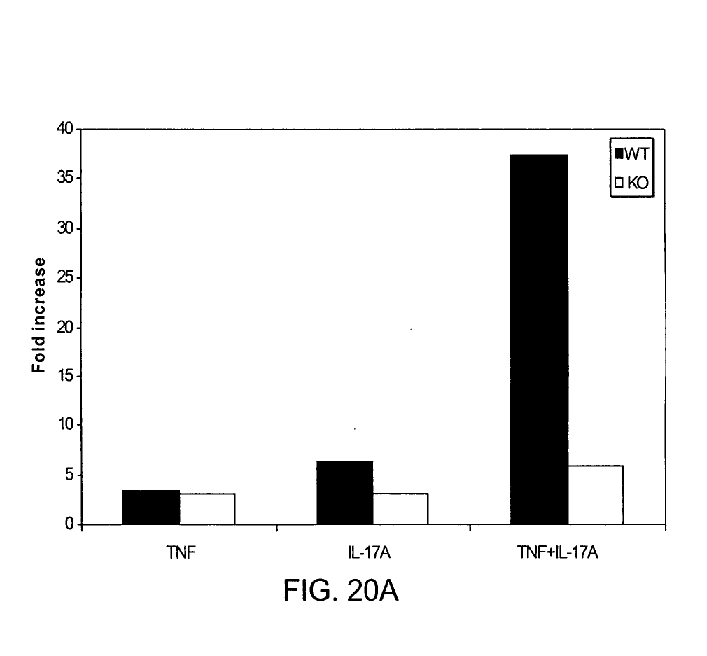

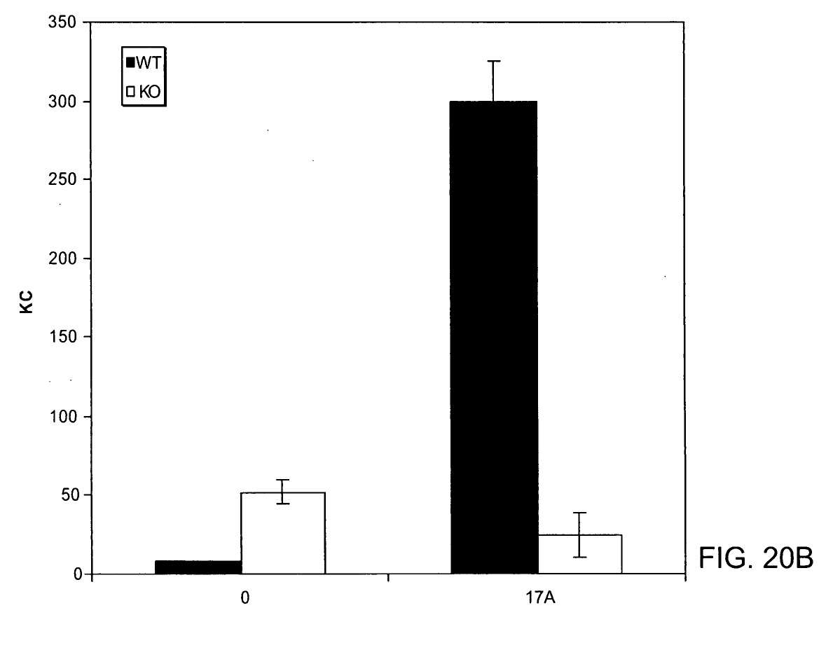

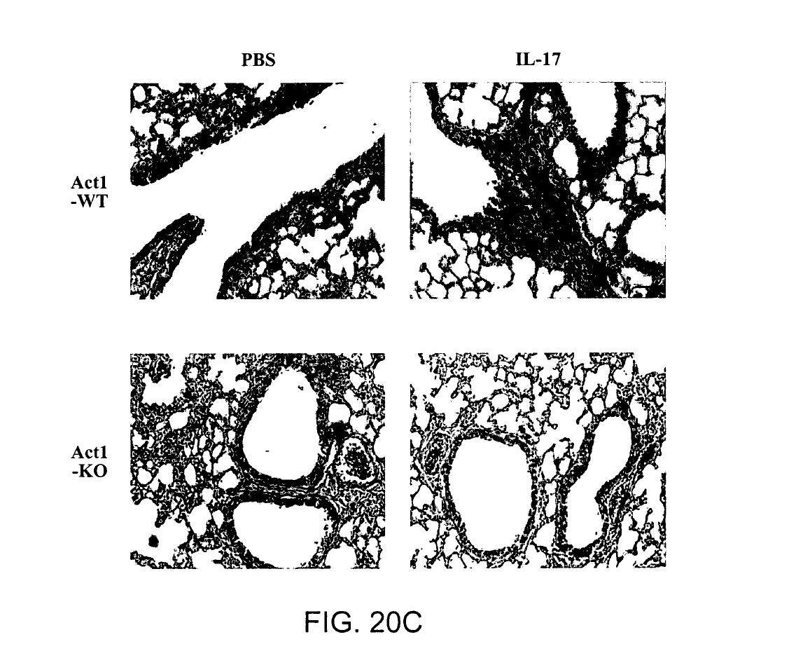

To examine if Act1 is required for IL-17 mediated inflammatory response, TNF, IL-17 and combination of IL-17 and TNF were injected intraperitoneally into Act1 deficient and wild-type control mice (FIG. 20A), and KC induction in the serum was measured 4 h after injection. There was no difference in KC induction upon TNF treatment. However, KC induction was much greater in Act1 deficient mice than in wild-type control mice. Epithelium-specific K18Cre Act1 deficient mice were also challenged intranasally and with IL-17. KC level was significantly lower in the bronchoalveolar lavage of K18Cre Act1 deficient mice compared to wild-type control mice (FIG. 20B). In addition, there was more inflammation observed in the lung tissue of K18Cre wild-type control mice than in the lung tissue of K18Cre Act1 deficient mice (FIG. 20C). These results indicates that Act1 indeed functions in vivo as a key component of IL-17 signaling.

Act1 is Required for IL-17 Mediated IκB Degradation but not for MAPK Activation

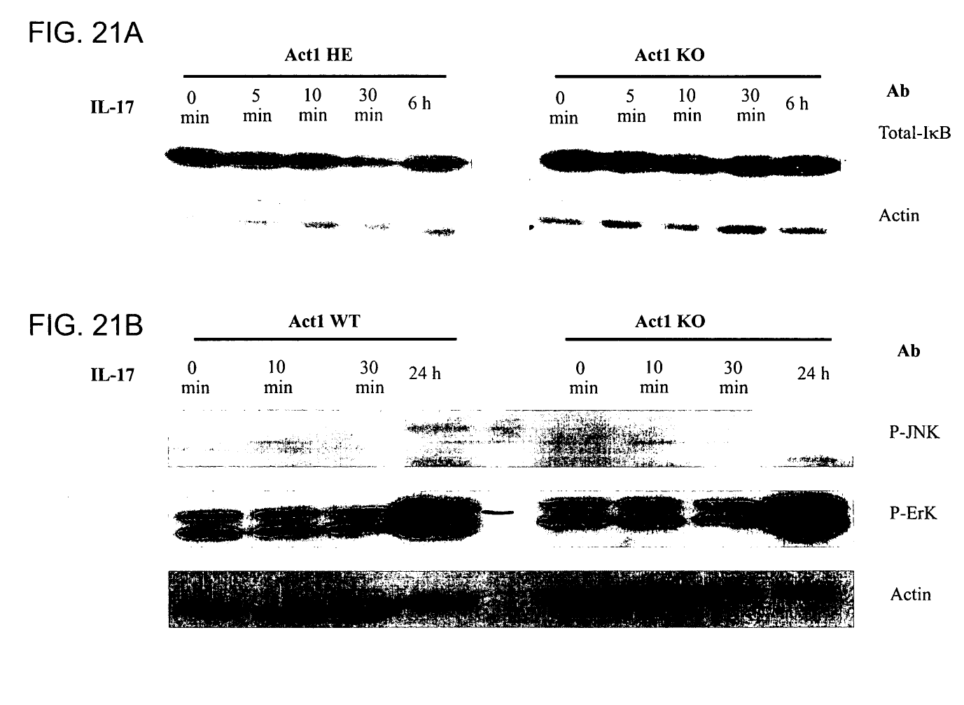

To determine which pathway Act1 is involve in IL-17 signaling, IκB degradation, phospho-JNK, phosphor-ERK and phospho-p38 was checked in both Act1 deficient and wild-type control MEFs upon IL-17 stimulation. After 30 min treatment, IL-17 could induce IκB degradation in wild-type MEFs but not in Act1 deficient MEFs (FIG. 21A). Therefore, it is likely that Act1 regulates IL-17 mediated NFκB activation. IL-17 could not induce JNK and ERK activation upon short-time treatment as indicated by phospho-JNK and phospho-ERK levels. However, after long-time (24 h) treatment, JNK and ERK were greatly activated in both Act1 deficient and wild-type MEFs (FIG. 21B). No detectable phopho-p38 was observed in both types of MEFs (data not shown).

IL-17 Receptor Interacts with Act1 and Other Signaling Molecules

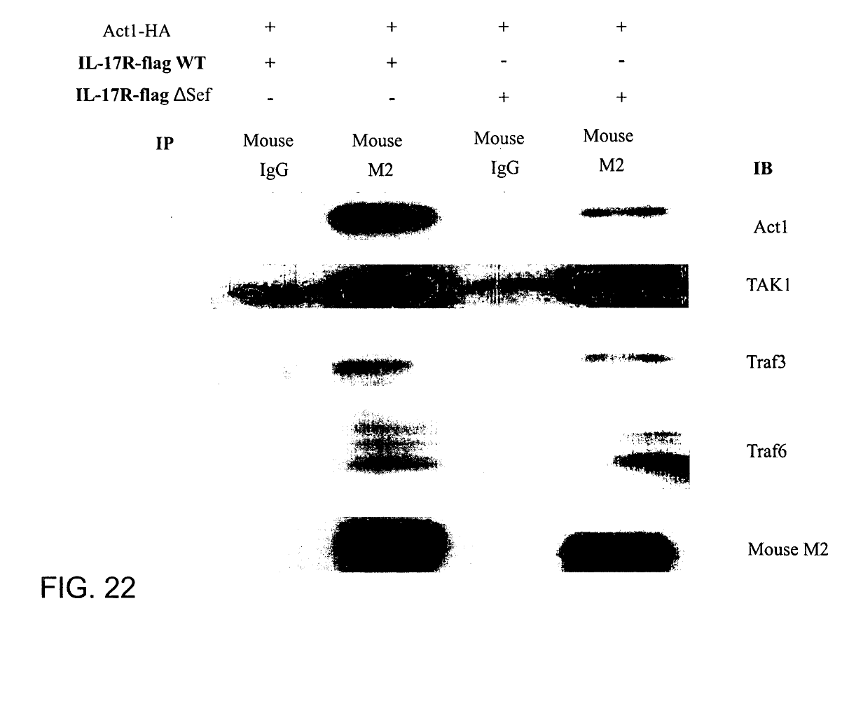

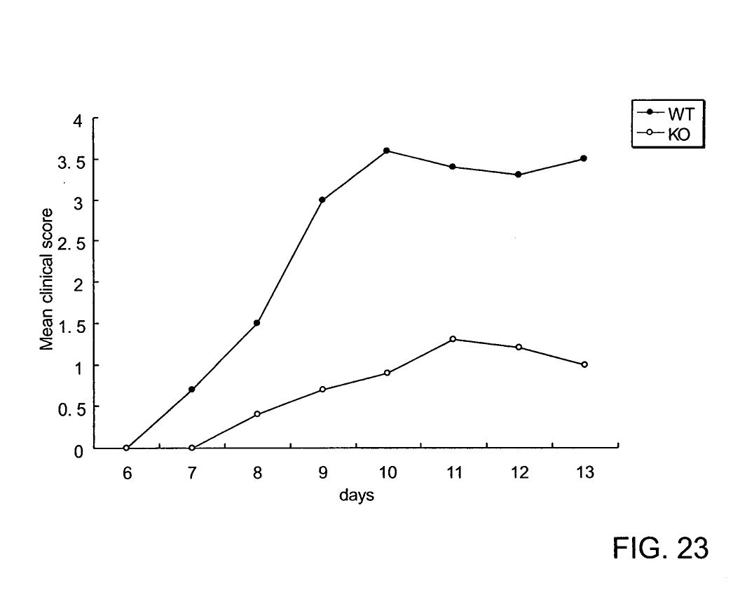

Although IL-17 receptor (IL-17R) has been cloned years ago, the detailed signaling mechanism is still poorly understood. It has been reported that both IL-17 receptor and Act1 contain a protein domain called SEF, which has been proposed to be responsible for homotypical protein interactions. To elucidate the IL-17 signaling pathway and place Act1 in this biological context, coimmunoprecipation experiments in 293 cells were conducted using full-length IL-17R and a SEF-deletion IL-17 R mutant-ΔSef (FIG. 22). Compared to full-length IL-17R, much less Act1 was associated with ΔSef, suggesting the interaction between IL-17R and Act1 is SEF-dependent. It was also observed that the IL-17R interacted with TAK1, Traf3 and some modified forms of Traf6 in a SEF-independent manner.

All references cited herein are incorporated by reference herein in their entirety.

While this invention has been particularly shown and described with references to preferred embodiments thereof, it will be understood by those skilled in the art that various changes in form and details may be made therein without departing from the scope of the invention encompassed by the appended claims.

1. A transgenic non-human mammal whose genome comprises a disruption of an Act1 gene.

2. The transgenic non-human mammal of claim 1 wherein the transgenic non-human mammal has lymphoid system defects comprising lymphadenopathy, hypergammaglobulinemia and production of autoantibodies.

3. The transgenic non-human mammal of claim 1 wherein the disruption of the Act1 gene is in exon 2 of the Act1 gene.

4. The transgenic non-human mammal of claim 3 wherein the disruption of the Act1 gene comprises a deletion of exon 2 of the Act1 gene.

5. The transgenic non-human mammal of claim 4 wherein an Act1 gene targeting vector is used to delete exon 2 of the Act1 gene.

6. The transgenic non-human mammal of claim 5 wherein the Act1 gene targeting vector comprises in a 5′ to 3′ order: a first intron of the Act1 gene—a recombination site—a marker gene—exon 2 of the Act1 gene—a recombination site—a second intron of the Act1 gene.

7. The transgenic non-human mammal of claim 6 wherein the recombination site is a loxP site and the marker gene is a Neo gene.

8. The transgenic non-human mammal of claim 1 wherein the mammal is a mouse.

9. The transgenic mouse of claim 8 wherein the transgenic mouse has lymphoid system defects comprising lymphadenopathy, hypergammaglobulinemia and production of autoantibodies.

10. The transgenic mouse of claim 8 wherein the disruption of the Act1 gene is in exon 2 of the Act 1 gene.

11. The transgenic mouse of claim 10 wherein the disruption of the Act1 gene comprises a deletion of exon 2 of the Act1 gene.

12. The transgenic mouse of claim 11 wherein an Act1 gene targeting vector is used to delete exon 2 of the Act1 gene.

13. The method of claim 12 wherein the Act1 gene targeting vector comprises in a 5′ to 3′ order: a first intron of the Act1 gene—a recombination site—a marker gene—exon 2 of the Act1 gene—a recombination site—a second intron of the Act1 gene.

14. The method of claim 13 wherein the recombination site is a loxP site and the marker gene is a Neo gene.

15. A method of producing a transgenic non-human mammal whose genome comprises a homozygous disruption of an Act 1 gene comprising:
a) introducing a targeting vector which disrupts the Act1 gene in an embryonic stem cell, thereby producing a transgenic embryonic stem cell with a disrupted Act1 gene;
b) introducing the transgenic embryonic stem cell into a blastocyte, thereby forming a chimeric blastocyte; and
c) introducing the chimeric blastocyte into the uterus of a pseudo-pregnant non-human mammal under conditions in which the pseudo-pregnant non-human mammal gives birth to transgenic non-human mammals whose genome comprises a heterozygous disruption of the Act1 gene;
d) breeding the transgenic non-human mammals of c) under conditions in which a transgenic non-human mammal whose genome comprises a homozygous disruption the Act 1 gene is produced.

16. The method of claim 15 wherein the transgenic non-human mammal has lymphoid system defects comprising lymphadenopathy, hypergammaglobulinemia and production of autoantibodies.

17. The method of claim 15 wherein the targeting vector causes disruption of exon 2 of the Act 1 gene.

18. The method of claim 17 wherein the targeting vector causes deletion of exon 2 of the Act1 gene.

19. The method of claim 18 wherein the targeting vector comprises in a 5′ to 3′ order a first intron of the Act1 gene—a recombination site—a marker gene—exon 2 of the Act1 gene—a recombination site—a second intron of the Act1 gene.

20. The method of claim 19 wherein the recombination site is a loxP site and the marker gene is a Neo gene.

21. A transgenic non-human mammal produced by the method of claim 15.

22. An isolated cell having a genome comprising a disruption of an Act1 gene, wherein the cell is isolated from the transgenic non-human mammal comprising a disruption of the Act1 gene of claim 1.

23. The isolated cell of claim 22 wherein the transgenic non-human mammal has lymphoid system defects comprising lymphadenopathy, hypergammaglobulinemia and production of autoantibodies.

24. The isolated cell of claim 22 wherein the disruption of the Act1 gene is in exon 2 of the Act1 gene.

25. The isolated cell of claim 24 wherein the disruption of the Act1 gene comprises a deletion of exon 2 of the Act1 gene.

26. The isolated cell of claim 25 wherein an Act1 gene targeting vector is used to delete exon 2 of the Act1 gene.

27. The isolated cell of claim 26 wherein the Act1 gene targeting vector comprises in a 5′ to 3′ order: a first intron of the Act1 gene—a recombination site—a marker gene—exon 2 of the Act1 gene—a recombination site—a second intron of the Act1 gene.

28. The isolated cell of claim 27 wherein the recombination site is a loxP site and the marker gene is a Neo gene.

29. The transgenic non-human mammal whose genome comprises a disruption of an Act1 gene of claim 1 wherein the disruption of the Act1 gene is specific to B cells of the transgenic non-human mammal.

30. The transgenic non-human mammal of claim 29 wherein the transgenic non-human mammal has lymphoid system defects comprising lymphadenopathy, hypergammaglobulinemia and production of autoantibodies.

31. The transgenic non-human mammal of claim 29 wherein the disruption of the Act1 gene is in exon 2 of the Act 1 gene.

32. The transgenic non-human mammal of claim 31 wherein the disruption of the Act1 gene comprises a deletion of exon 2 of the Act1 gene.

33. The transgenic non-human mammal whose genome comprises a disruption of an Act1 gene of claim 1 wherein the disruption of the Act1 gene is specific to epithelial cells of the transgenic non-human mammal.

34. The transgenic non-human mammal of claim 1 whose genome further comprises a disruption of a CD40.

35. The transgenic non-human mammal of claim 34 wherein the transgenic non-human mammal has enlarged lymph nodes and an enlarged spleen.

36. The transgenic non-human mammal of claim 1 whose genome further comprises a disruption of a BAFF gene.

37. The transgenic non-human mammal of claim 36 wherein the transgenic non-human mammal has enlarged lymph nodes and an enlarged spleen.

38. A transgenic non-human mammal whose genome comprises a heterozygous disruption of an Act1 gene wherein disruption of the Act1 gene in a homozygous states results in a transgenic mouse having lymphoid system defects comprising lymphadenopathy, hypergammaglobulinemia and production of autoantibodies.

39. A method of producing antibodies which specifically bind to an antigen comprising introducing the antigen into the transgenic non-human mammal whose genome comprises a disruption of an Act1 gene of claim 1 under conditions in which antibodies which specifically bind to the antigen are produced in the mammal.

40. The method of claim 39 further comprising isolating the antibodies which specifically bind to the antigen from the transgenic non-human mammal.

41. The method of claim 39 wherein the antigen is selected from the group consisting of: a T cell independent antigen and a T cell dependent antigen.

42. The method of claim 39 wherein the antigen does not initiate a strong antibody response in a wild type mammal.

43. The method of claim 39 wherein the antibodies have a higher affinity for the antigen when compared to antibodies made in a wild type non-human mammal.

44. The method of claim 39 wherein the transgenic non-human mammal has lymphoid system defects comprising lymphadenopathy, hypergammaglobulinemia and production of autoantibodies.

45. Antibodies produced by the method of claim 39.

46. A method of producing a hybridoma which expresses a monoclonal antibody that is specifically directed to an antigen comprising
a) introducing the antigen into the transgenic non-human mammal whose genome comprises a disruption of an Act 1 gene of claim 1, under conditions in which antibodies which specifically bind to the antigen are produced in the mammal;
b) isolating B cells from the mammal;
c) selecting a B cell from the B cells of b) which expresses an antibody that specifically recognizes the antigen;
d) fusing the B cell of c) with an immortal cell,
thereby producing a hybridoma which expresses a monoclonal antibody that is specifically directed to the antigen.

47. The method of claim 46 further comprising isolating the hybridoma.

48. A hybridoma produced by the method of claim 46.

49. A method of producing a monoclonal antibody that is specifically directed to an antigen comprising
a) introducing the antigen into the transgenic non-human mammal whose genome comprises a disruption of an Act1 gene of claim 1, under conditions in which antibodies which specifically bind to the antigen are produced in the mammal;
b) isolating B cells from the mammal;
c) selecting a B cell from the B cells of b) which expresses an antibody that specifically recognizes the antigen;
d) fusing the B cell of c) with an immortal cell, thereby producing a hybridoma which expresses a monoclonal antibody that is specifically directed to the antigen;
e) maintaining the hybridoma of d) under conditions in which the monoclonal antibody is expressed,
thereby producing a monoclonal antibody that is specifically directed to an antigen.

50. The method of claim 49 further comprising isolating the monoclonal antibody.

51. A monoclonal antibody produced by the method of claim 49.

52. A method of identifying an agent to treat or prevent systemic lupus erythematosus (SLE) comprising:
a) administering to the transgenic non-human mammal whose genome comprises a disruption of an Act1 gene of claim 1 an agent to be assessed;
b) determining the ability of the agent to treat or prevent SLE in the transgenic non-human mammal,
wherein if the agent treats or prevents SLE in the transgenic non-human mammal, then the agent is an agent which can be used to treat or prevent SLE.

53. A method of identifying an agent to treat or prevent Sjögren's syndrome comprising:
c) administering to the transgenic non-human mammal whose genome comprises a disruption of an Act1 gene of claim 1 an agent to be assessed;
d) determining the ability of the agent to treat or prevent Sjögren's syndrome in the transgenic non-human mammal,
wherein if the agent treats or prevents Sjögren's syndrome in the transgenic non-human mammal, then the agent is an agent which can be used to treat or prevent Sjögren's syndrome.

54. A method of identifying an agent to treat or prevent cancer comprising:
e) administering to the transgenic non-human mammal whose genome comprises a disruption of an Act1 gene of claim 1 an agent to be assessed;
f) determining the ability of the agent to treat or prevent cancer in the transgenic non-human mammal,
wherein if the agent treats or prevents cancer in the transgenic non-human mammal, then the agent is an agent which can be used to treat or prevent cancer.

55. A vector comprising in a 5′ to 3′ order: a first intron of the Act1 gene—a recombination site—a marker gene—exon 2 of the Act1 gene—a recombination site—a second intron of the Act1 gene.

56. The vector of claim 55 wherein the recombination site is a loxP site and the marker gene is a Neo gene.


SEQUENCE LISTING (8 sequences)

SEQ ID NO: 1 - DNA, 23 bp, Artificial Sequence
  synthetic duplex for human Act1
aagctcactcaaccctgaaactt

SEQ ID NO: 2 - protein, 5 aa, Unknown
  TRAF binding site in Act1, residues 38-42
EEESE

SEQ ID NO: 3 - protein, 5 aa, Unknown
  TRAF binding site in Act1, residues 333-337
EERPA

SEQ ID NO: 4 - protein, 21 aa, Unknown
  myelin oligodendrocyte glycoprotein 33-35 peptide (MOG 33-35)
MEVGWYRSPFSRVVHLYRNGK

SEQ ID NO: 5 - DNA, 1725 bp, Unknown
  human Act1
atgcctcctcagcttcaagaaactagaatgaaccgaagcattcctgtggaggttgatgaatcagaaccatacccaagtcagttgctgaaaccaatcccagaatattccccggaagaggaatcagaaccacctgctccaaatataaggaacatggcacccaacagcttgtctgcacccacaatgcttcacaattcctccggagacttttctcaagctcactcaaccctgaaacttgcaaatcaccagcggcctgtatcccggcaggtcacctgcctgcgcactcaagttctggaggacagtgaagacagtttctgcaggagacacccaggcctgggcaaagctttcccttctgggtgctctgcagtcagcgagcctgcgtctgagtctgtggttggagccctccctgcagagcatcagttttcatttatggaaaaacgtaatcaatggctggtatctcagctttcagcggcttctcctgacactggccatgactcagacaaatcagaccaaagtttacctaatgcctcagcagactccttgggcggtagccaggagatggtgcaacggccccagcctcacaggaaccgagcaggcctggatctgccaaccatagacacgggatatgattcccagccccaggatgtcctgggcatcaggcagctggaaaggcccctgcccctcacctccgtgtgttacccccaggacctccccagacctctcaggtccagggagttccctcagtttgaacctcagaggtatccagcatgtgcacagatgctgcctcccaatctttccccacatgctccatggaactatcattaccattgtcctggaagtcccgatcaccaggtgccatatggccatgactaccctcgagcagcctaccagcaagtgatccagccggctctgcctgggcagcccctgcctggagccagtgtgagaggcctgcaccctgtgcagaaggttatcctgaattatcccagcccctgggaccacgaagagaggcccgcacagagagactgctcctttccggggcttccaaggcaccaggaccagccacatcaccagccacctaatagagctggtgctcctggggagtccttggagtgccctgcagagctgagaccacaggttccccagcctccgtccccagctgctgtgcctagaccccctagcaaccctccagccagaggaactctaaaaacaagcaatttgccagaagaattgcggaaagtctttatcacttattcgatggacacagctatggaggtggtgaaattcgtgaactttttgttggtaaatggcttccaaactgcaattgacatatttgaggatagaatccgaggcattgatatcattaaatggatggagcgctaccttagggataagaccgtgatgataatcgtagcaatcagccccaaatacaaacaggacgtggaaggcgctgagtcgcagctggacgaggatgagcatggcttacatactaagtacattcatcgaatgatgcagattgagttcataaaacaaggaagcatgaatttcagattcatccctgtgctcttcccaaatgctaagaaggagcatgtgcccacctggcttcagaacactcatgtctacagctggcccaagaataaaaaaaacatcctgctgcggctgctgagagaggaagagtatgtggctcctccacgggggcctctgcccacccttcaggtggttcccttgtga

SEQ ID NO: 6 - protein, 574 aa, Unknown
  human Act1
MPPQLQETRMNRSIPVEVDESEPYPSQLLKPIPEYSPEEESEPPAPNIRNMAPNSLSAPTMLHNSSGDFSQAHSTLKLANHQRPVSRQVTCLRTQVLEDSEDSFCRRHPGLGKAFPSGCSAVSEPASESVVGALPAEHQFSFMEKRNQWLVSQLSAASPDTGHDSDKSDQSLPNASADSLGGSQEMVQRPQPHRNRAGLDLPTIDTGYDSQPQDVLGIRQLERPLPLTSVCYPQDLPRPLRSREFPQFEPQRYPACAQMLPPNLSPHAPWNYHYHCPGSPDHQVPYGHDYPRAAYQQVIQPALPGQPLPGASVRGLHPVQKVILNYPSPWDHEERPAQRDCSFPGLPRHQDQPHHQPPNRAGAPGESLECPAELRPQVPQPPSPAAVPRPPSNPPARGTLKTSNLPEELRKVFITYSMDTAMEVVKFVNFLLVNGFQTAIDIFEDRIRGIDIIKWMERYLRDKTVMIIVAISPKYKQDVEGAESQLDEDEHGLHTKYIHRMMQIEFIKQGSMNFRFIPVLFPNAKKEHVPTWLQNTHVYSWPKNKKNILLRLLREEEYVAPPRGPLPTLQVVPL

SEQ ID NO: 7 - DNA, 1668 bp, Unknown
  murine Act1
atgaaccgaagcattcccgtggaggttgatgaatcagaaccattcccgagtcaattgctgaaaccgatcccagaatactctccagaagaggaattggaaccacctgcccccaatacaaggaacatggcacccagcagcttgtctgtgctccagtgccctcccctgaaactggcaaaccaccagcctgtgtcacaacaggtcacctgcctgcgagctaaagtcctggaggaaggtgaagccagtttctttagaaggcaccctgaacttggcaaagatatttcctcatgttcctctggagccagcgagcctgagtctgagctcggagccctccctccagagcatcggttcacacttacggaaaaacgcaatagatggctgggatctcagctttcagcagcttctcctgacactggccatgaatcagacaaatcagacccaagtttacctaatgctttggcagactccttcagtggtggccaagagatgatgccccggccccggccccggcctgggccccacaggcacagggcagccccagatgtgcccacgatagacactggctatgattcccagccccaagatgtcctgggcatcaggcagttggagaggccattgcccctcacctcctcgtgttacctgcaggacctccctgggcctctgaggtccagggaattgccacctcagtttgaacttgagaggtatccaatgaacgcccagctgctgccgccccatccttccccacaggccccatggaactgtcagtactactgccccggagggccctaccaccaccaggtgccacacggccatggctaccctccagcagcagcctaccagcaagtactccagcctgctctgcctgggcaggtccttcctggggcaagggcaagaggcccacgccctgtgcagaaggtcatcctgaatgactccagcccccaagaccaagaagagagacctgcacagagagacttctctttcccgaggctcccgagggaccagctctaccgcccaccatctaatggagtggaagcccctgaggagtccttggaccttcctgcagagctgagaccacatggtccccaggctccatccctagctgccgtgcctagaccccctagcaaccccttagcccgaggaactctaagaaccagcaatttgccagaagaattacggaaagtctttatcacttattctatggacacagccatggaggtggtgaaatttgtgaactttctgttggtgaacggcttccaaactgcgattgacatatttgaggatagaatccggggtattgatatcattaaatggatggagcgctatcttcgagataagacagtgatgataatcgtagcaatcagccccaaatacaaacaggatgtggaaggcgctgagtcgcagctggacgaggacgagcatggcttacatactaagtacattcatcggatgatgcagattgagttcataagtcagggaagcatgaacttcagattcatccctgtgctcttcccaaatgccaagaaggagcatgtgccgacctggcttcagaacactcatgtttacagctggcccaagaataagaaaaacatcctgctgcggctgctcagggaggaagagtatgtggctcctccccgaggccctctgcccacccttcaggtggtacccttgtga

SEQ ID NO: 8 - protein, 555 aa, Unknown
  murine Act1
MNRSIPVEVDESEPFPSQLLKPIPEYSPEEELEPPAPNTRNMAPSSLSVLQCPPLKLANHQPVSQQVTCLRAKVLEEGEASFFRRHPELGKDISSCSSGASEPESELGALPPEHRFTLTEKRNRWLGSQLSAASPDTGHESDKSDPSLPNALADSFSGGQEMMPRPRPRPGPHRHRAAPDVPTIDTGYDSQPQDVLGIRQLERPLPLTSSCYLQDLPGPLRSRELPPQFELERYPMNAQLLPPHPSPQAPWNCQYYCPGGPYHHQVPHGHGYPPAAAYQQVLQPALPGQVLPGARARGPRPVQKVILNDSSPQDQEERPAQRDFSFPRLPRDQLYRPPSNGVEAPEESLDLPAELRPHGPQAPSLAAVPRPPSNPLARGTLRTSNLPEELRKVFITYSMDTAMEVVKFVNFLLVNGFQTAIDIFEDRIRGIDIIKWMERYLRDKTVMIIVAISPKYKQDVEGAESQLDEDEHGLHTKYIHRMMQIEFISQGSMNFRFIPVLFPNAKKEHVPTWLQNTHVYSWPKNKKNILLRLLREEEYVAPPRGPLPTLQVVPL